(19) United States
(12) Patent Application Publication
(10) Pub. No.: US 2025/0281577 A1
(43) Pub. Date: Sep. 11, 2025

(54) USE OF GLP-1 RECEPTOR AGONISTS TO TREAT IMPULSE-CONTROL DISORDERS

(71) Applicants: Jon E. Grant, Henderson, NV (US); David H. Zald, Piscataway, NY (US); Suck Won Kim, Henderson, NV (US)

(72) Inventors: Jon E. Grant, Henderson, NV (US); David H. Zald, Piscataway, NY (US); Suck Won Kim, Henderson, NV (US)

(21) Appl. No.: 18/596,475

(22) Filed: Mar. 5, 2024

(51) Int. Cl. A61K 38/26

(52) U.S. Cl. CPC A61K 38/26

(57) ABSTRACT

The disclosure relates to a therapeutic method of treating an impulse-control disorder comprising administering to an individual afflicted with an impulse-control disorder an amount of at least one glucagon-like-peptide-1 (GLP-1) effective to reduce or eliminate at least one of the symptoms of the impulse-control disorder.

### BACKGROUND

Intermittent explosive disorder, kleptomania, pyromania, pathological gambling, and trichotillomania can be grouped as impulse-control disorders. However, commentators have recently questioned whether or not trichotillomania is properly classified as an impulse-control disorder, and some have urged that it should be classified as an obsessive-compulsive disorder (OCD). See for example, V. G. Carrion, J. Clin. Psychopharmacol., 15, 444 (1995); D. J. Stern et al., J. Clin. Psychiatry, 56, (suppl. 4) 28 (1995) and S. E. Swedo et al., Psychiatr. Clin. North Am., 15, 777 (1992). Compulsive shopping and the other descriptive diagnoses describe problematic behaviors that are triggered by uncontrollable underlying urges. Urges, however, have not been defined as the primary problem. Instead, they have been viewed as one of the symptoms of each disorder.

Historically, impulse-control disorders (ICD) have been considered refractory to known pharmacological or psychotherapeutic treatments. Therefore, a continuing need exists for agents which will be effective to treat (eliminate or reduce) these symptoms associated with ICD's.

### SUMMARY

Although ICDs have been treated with a variety of therapeutics, they have not been treated with glucagon-like peptide 1 (GLP-1) receptor agonists. The disclosure therefore relates to a therapeutic method of treating an impulse-control disorder comprising administering to an individual afflicted with an impulse-control disorder an amount of at least one glucagon-like-peptide-1 (GLP-1) effective to reduce or eliminate at least one of the symptoms of the impulse-control disorder.

### DESCRIPTION

Reference will now be made in detail to certain embodiments of the disclosed subject matter. While the disclosed subject matter will be described in conjunction with the enumerated claims, it will be understood that the exemplified subject matter is not intended to limit the claims to the disclosed subject matter.

The disclosure relates to a method of treating an impulse-control disorder comprising administering to an individual afflicted with an impulse-control disorder an amount of at least one glucagon-like peptide 1 (GLP-1) receptor agonist effective to reduce or eliminate at least one of the symptoms of the impulse-control disorder. Examples of impulse-control disorders include intermittent explosive disorder, kleptomania, pyromania, pathological gambling, compulsive shopping, and compulsive sexual behavior/disorder.

### Compulsive Sexual Behavior/Disorder

Compulsive sexual behavior is sometimes called hypersexuality or sexual addiction. It is an intense focus on sexual fantasies, urges or behaviors that cannot be controlled. This causes distress and problems in a person's health, job, relationships or other parts of their life.

Compulsive sexual behavior may involve different kinds of commonly enjoyable sexual experiences. Examples include masturbation, sexual arousal by using a computer to communicate, multiple sexual partners, use of pornography or paying for sex. But when these sexual behaviors become a major, constant focus in a person's life, are difficult to control, cause problems in a person's life, or are harmful to them or others, that is likely compulsive sexual behavior.

### Intermittent Explosive Disorder

The essential feature of Intermittent Explosive Disorder is the occurrence of discrete episodes of failure to resist aggressive impulses that result in serious assaultive acts or destruction of property. The degree of aggressiveness expressed during an episode is grossly out of proportion to any provocation or precipitating psychosocial stressor. A diagnosis of Intermittent Explosive Disorder is made only after other mental disorders that might account for episodes of aggressive behavior have been ruled out (e.g., Antisocial Personality Disorder, Borderline Personality Disorder, a Psychotic Disorder, a Manic Episode, Conduct Disorder, or Attention Deficit/Hyperactivity Disorder). The aggressive episodes are not due to the direct physiological effects of a substance (e.g., an abused drug, a medication) or a general medical condition (e.g., head trauma, Alzheimer's disease). The individual may describe the aggressive episodes as “spells” or “attacks” in which the explosive behavior is preceded by a sense of tension or arousal and is followed immediately by a sense of relief. Later the individual may feel upset, remorseful, regretful, or embarrassed about the aggressive behavior.

### Kleptomania

The essential feature of Kleptomania is the recurrent failure to resist impulses to steal items even though the items are not needed for personal use or for their monetary value. The individual experiences a rising subjective sense of tension before the theft and feels pleasure, gratification, or relief when committing the theft. The stealing is not committed to express anger or vengeance, is not done in response to a delusion or hallucination, and is not better accounted for by Conduct Disorder, a Manic Episode, or Antisocial Personality Disorder. The objects are stolen despite the fact that they are typically of little value to the individual, who could have afforded to pay for them and often gives them away or discards them. Occasionally the individual may hoard the stolen objects or surreptitiously return them. Although individuals with this disorder will generally avoid stealing when immediate arrest is probable (e.g., in full view of a police officer), they usually do not preplan the thefts or fully take into account the chances of apprehension. The stealing is done without assistance from, or collaboration with, others.

### Pyromania

The essential feature of Pyromania is the presence of multiple episodes of deliberate and purposeful fire setting. Individuals with this disorder experience tension or affective arousal before setting a fire. There is a fascination with, interest in, curiosity about, or attraction to fire and its situational contexts (e.g., paraphernalia, uses, consequences). Individuals with this disorder are often regular “watchers” at fires in their neighborhoods, may set off false alarms, and derive pleasure from institutions, equipment, and personnel associated with fire. They may spend time at the local fire department, set fires to be affiliated with the fire department, or even become firefighters. Individuals with this disorder experience pleasure, gratification, or a release of tension when setting the fire, witnessing its effects, or participating in its aftermath. The fire setting is not done for monetary gain, as an expression of sociopolitical ideology, to conceal criminal activity, to express anger or vengeance, to improve one's living circumstances, or in response to a delusion or a hallucination. The fire setting does not result from impaired judgment (e.g., in dementia, Mental Retardation, or Substance Intoxication).

### Pathological Gambling

The essential feature of Pathological Gambling is persistent and recurrent maladaptive gambling behavior that disrupts personal, family, or vocational pursuits. The diagnosis is not made if the gambling behavior is better accounted for by a Manic Episode.

The individual may be preoccupied with gambling (e.g., reliving past gambling experiences, planning the next gambling venture, or thinking of ways to get money with which to gamble). Most individuals with Pathological Gambling say that they are seeking “action” (an aroused, euphoric state) even more than money. Increasingly larger bets, or greater risks, may be needed to continue to produce the desired level of excitement. Individuals with Pathological Gambling often continue to gamble despite repeated efforts to control, cut back, or stop the behavior. There may be restlessness or irritability when attempting to cut down or stop gambling. The individual may gamble as a way of escaping from problems or to relieve a dysphoric mood (e.g., feelings of helplessness, guilt, anxiety, depression). A pattern of “chasing” one's losses may develop, with an urgent need to keep gambling (often with larger bets or the taking of greater risks) to undo a loss or series of losses. The individual may abandon his or her gambling strategy and try to win back losses all at once. Although all gamblers may chase for short periods, it is the long-term chase that is more characteristic of individuals with Pathological Gambling. The individual may lie to family members, therapists, or others to conceal the extent of involvement with gambling. When the individual's borrowing resources are strained, the person may resort to antisocial behavior (e.g., forgery, fraud, theft, or embezzlement) to obtain money. The individual may have jeopardized or lost a significant relationship, job, or educational or career opportunity because of gambling. The individual may also engage in “bailout” behavior, turning to family or others for help with a desperate financial situation that was caused by gambling.

The diagnoses discussed hereinabove are excerpted from the Diagnostic and Statistical Manual of Mental Disorders, American Psychiatric Association, Washington D.C. (4th ed. 1994) at 609-621. Compulsive shopping shares many of the features of pathological gambling.

Examples of GLP-1 receptor agonists include tirzepatide, dulaglutide, exenatide (weekly and twice-daily), exenatide, semaglutide, liraglutide, and lixisenatide.

Examples of GLP-1 receptor agonists also include those disclosed in WO2023/198140, which is incorporated by reference as if fully set forth herein, falling within the general structure:

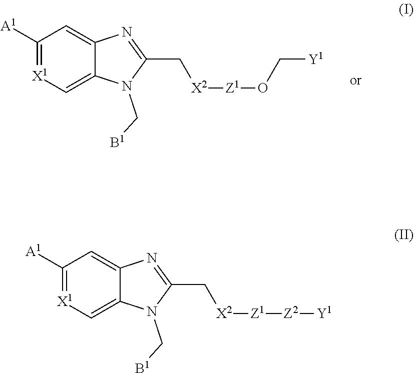

and pharmaceutically acceptable salts thereof; wherein:
A¹ is absent, alkyl, CN, five-membered heterocyclyl, —C(O)R¹, wherein R¹ is —OR² or —N(R²)₂,wherein each R² is independently H or alkyl;
X¹ is N or CH;
B¹ is a four- or five-membered heterocycloalkyl group (e.g., an oxetanyl group or a furanyl group);
X² is a five- or six-membered heterocycloalkyl group;
Z¹ is a six-membered heteroaryl group;
Y¹ is aryl or heteroaryl; and
Z² is an aryl group of the formula:

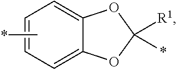

wherein R¹ is C₁₋₆-alkyl, such as the group:

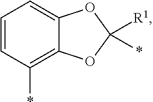

wherein the bonding arrangement to Z¹ and Y¹ is:

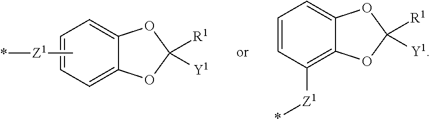

The asterisk indicates a point of attachment to another group.

Examples of compounds of the formulae (1) and (11) include, but are not limited to, the following:

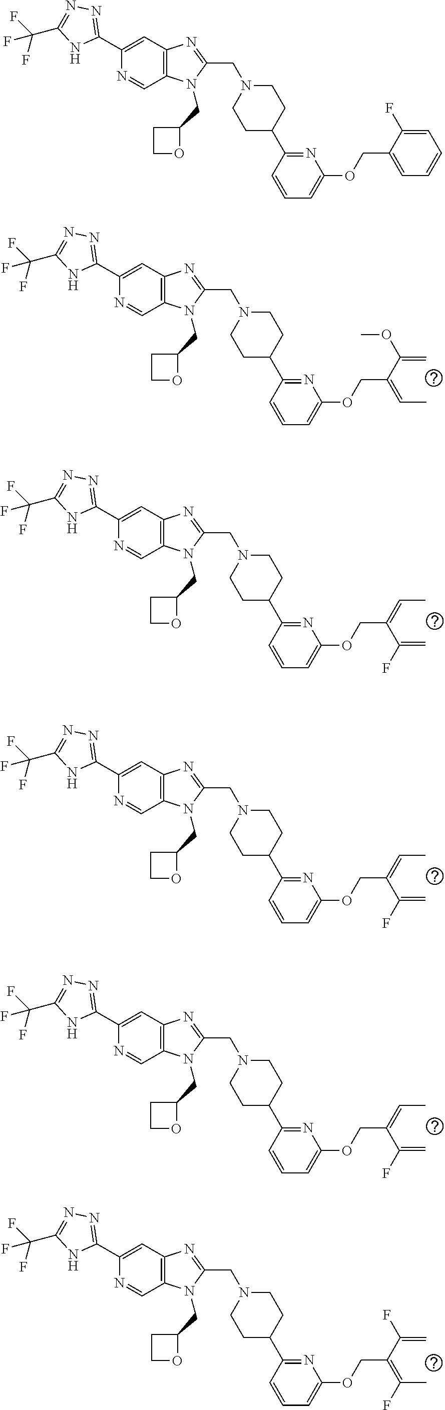

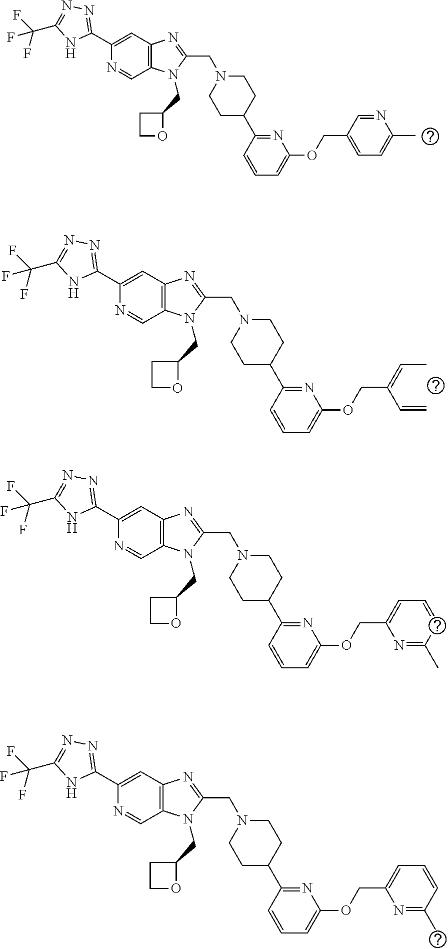 [SMILES: Cc1ccc(COc2cccc(C3CCN(Cc4nc5cc(-c6nnc(C(F)(F)F)[nH]6)ncc5n4C[C@@H]4CCO4)CC3)n2)cn1 ; C=C/C(=C\C)COc1cccc(C2CCN(Cc3nc4cc(-c5nnc(C(F)(F)F)[nH]5)ncc4n3C[C@@H]3CCO3)CC2)n1 ; Cc1cccc(COc2cccc(C3CCN(Cc4nc5cc(-c6nnc(C(F)(F)F)[nH]6)ncc5n4C[C@@H]4CCO4)CC3)n2)n1 ; Cc1cccc(COc2cccc(C3CCN(Cc4nc5cc(-c6nnc(C(F)(F)F)[nH]6)ncc5n4C[C@@H]4CCO4)CC3)n2)n1]

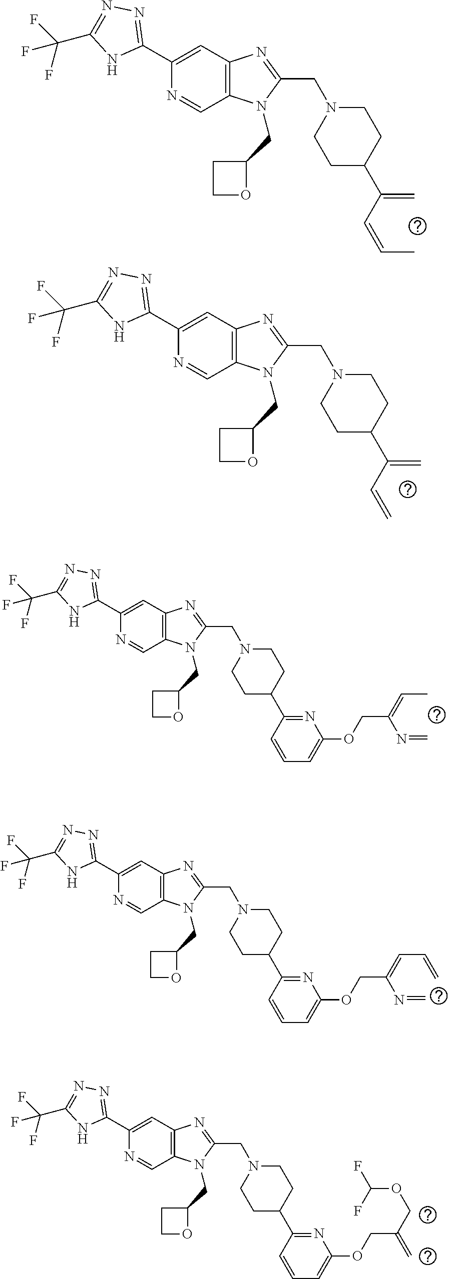 [SMILES: C=C(/C=C\C)C1CCN(Cc2nc3cc(-c4nnc(C(F)(F)F)[nH]4)ncc3n2C[C@@H]2CCO2)CC1 ; C=CC(=C)C1CCN(Cc2nc3cc(-c4nnc(C(F)(F)F)[nH]4)ncc3n2C[C@@H]2CCO2)CC1 ; C=N/C(=C\C)COc1cccc(C2CCN(Cc3nc4cc(-c5nnc(C(F)(F)F)[nH]5)ncc4n3C[C@@H]3CCO3)CC2)n1 ; C=C/C=C(/COc1cccc(C2CCN(Cc3nc4cc(-c5nnc(C(F)(F)F)[nH]5)ncc4n3C[C@@H]3CCO3)CC2)n1)N=C ; C=C(COc1cccc(C2CCN(Cc3nc4cc(-c5nnc(C(F)(F)F)[nH]5)ncc4n3C[C@@H]3CCO3)CC2)n1)COC(F)F]

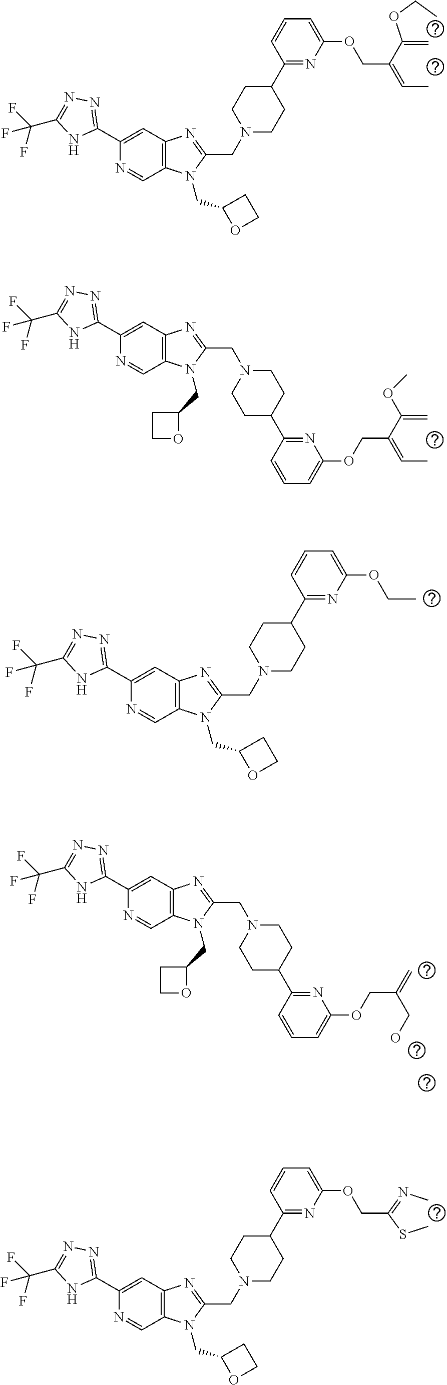 [SMILES: C=C(OCC)/C(=C\C)COc1cccc(C2CCN(Cc3nc4cc(-c5nnc(C(F)(F)F)[nH]5)ncc4n3C[C@@H]3CCO3)CC2)n1 ; C=C(OC)/C(=C\C)COc1cccc(C2CCN(Cc3nc4cc(-c5nnc(C(F)(F)F)[nH]5)ncc4n3C[C@@H]3CCO3)CC2)n1 ; CCOc1cccc(C2CCN(Cc3nc4cc(-c5nnc(C(F)(F)F)[nH]5)ncc4n3C[C@@H]3CCO3)CC2)n1 ; C=C(CO)COc1cccc(C2CCN(Cc3nc4cc(-c5nnc(C(F)(F)F)[nH]5)ncc4n3C[C@@H]3CCO3)CC2)n1 ; C/N=C(/COc1cccc(C2CCN(Cc3nc4cc(-c5nnc(C(F)(F)F)[nH]5)ncc4n3C[C@@H]3CCO3)CC2)n1)SC]

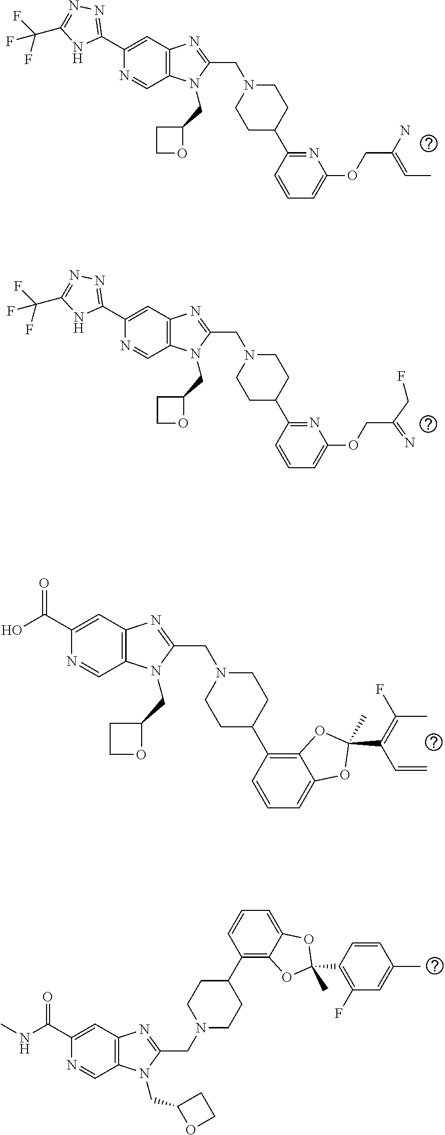

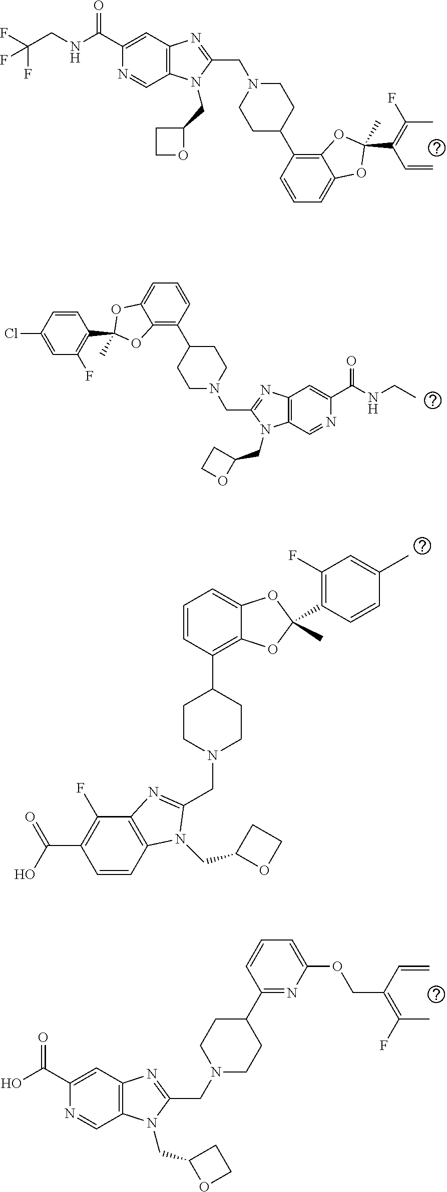 [SMILES: C=C/C(=C(\C)F)[C@@]1(C)Oc2cccc(C3CCN(Cc4nc5cc(C(=O)NCC(F)(F)F)ncc5n4C[C@@H]4CCO4)CC3)c2O1 ; CCNC(=O)c1cc2nc(CN3CCC(c4cccc5c4O[C@@](C)(c4ccc(Cl)cc4F)O5)CC3)n(C[C@@H]3CCO3)c2cn1 ; Cc1ccc([C@@]2(C)Oc3cccc(C4CCN(Cc5nc6c(F)c(C(=O)O)ccc6n5C[C@@H]5CCO5)CC4)c3O2)c(F)c1 ; C=C/C(COc1cccc(C2CCN(Cc3nc4cc(C(=O)O)ncc4n3C[C@@H]3CCO3)CC2)n1)=C(\C)F]

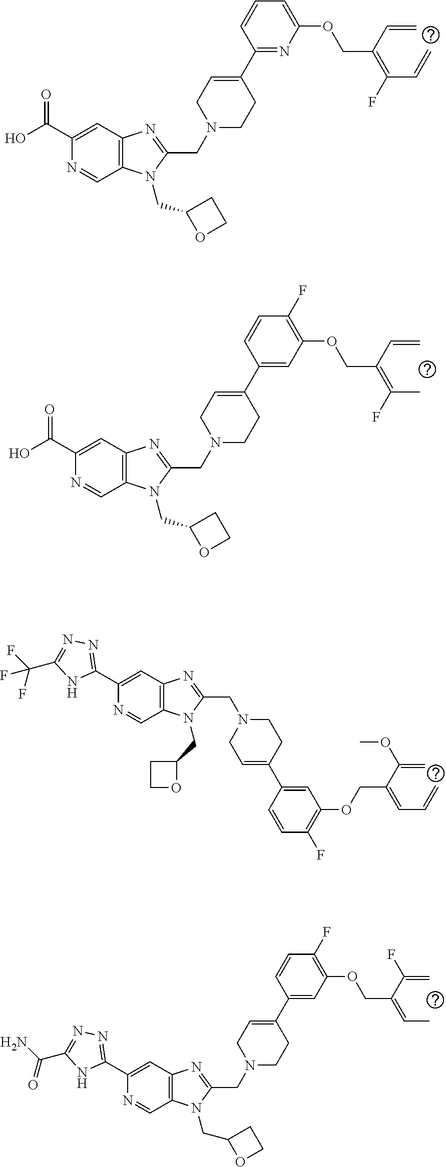 [SMILES: C=C/C(F)=C(\C=C)COc1cccc(C2=CCN(Cc3nc4cc(C(=O)O)ncc4n3C[C@@H]3CCO3)CC2)n1 ; C=C/C(COc1cc(C2=CCN(Cc3nc4cc(C(=O)O)ncc4n3C[C@@H]3CCO3)CC2)ccc1F)=C(\C)F ; C=C/C=C(/COc1cc(C2=CCN(Cc3nc4cc(-c5nnc(C(F)(F)F)[nH]5)ncc4n3C[C@@H]3CCO3)CC2)ccc1F)C(=C)OC ; C=C(F)/C(=C\C)COc1cc(C2=CCN(Cc3nc4cc(-c5nnc(C(N)=O)[nH]5)ncc4n3CC3CCO3)CC2)ccc1F]

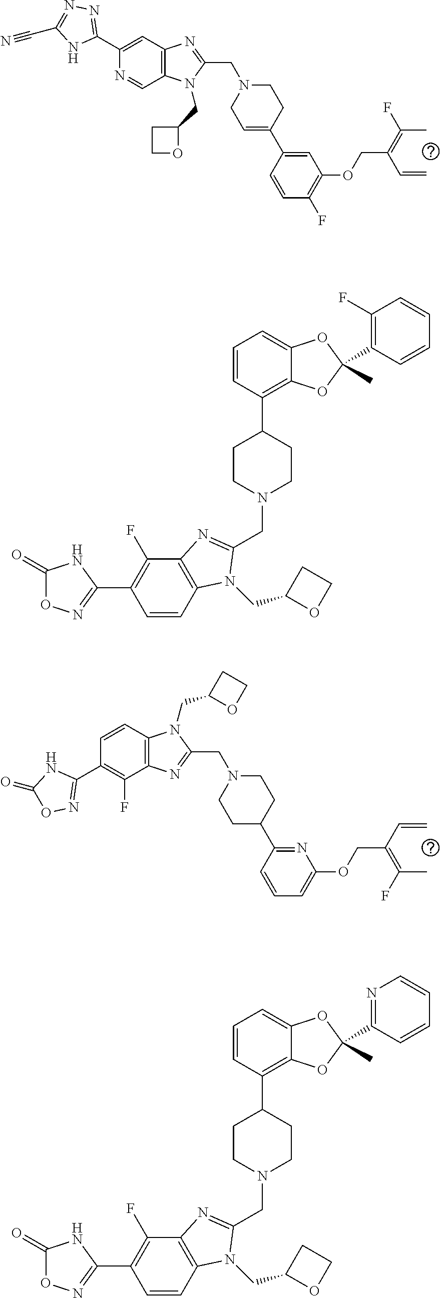 [SMILES: C=C/C(COc1cc(C2=CCN(Cc3nc4cc(-c5nnc(C#N)[nH]5)ncc4n3C[C@@H]3CCO3)CC2)ccc1F)=C(\C)F ; C[C@]1(c2ccccc2F)Oc2cccc(C3CCN(Cc4nc5c(F)c(-c6noc(=O)[nH]6)ccc5n4C[C@@H]4CCO4)CC3)c2O1 ; C=C/C(COc1cccc(C2CCN(Cc3nc4c(F)c(-c5noc(=O)[nH]5)ccc4n3C[C@@H]3CCO3)CC2)n1)=C(\C)F ; C[C@]1(c2ccccn2)Oc2cccc(C3CCN(Cc4nc5c(F)c(-c6noc(=O)[nH]6)ccc5n4C[C@@H]4CCO4)CC3)c2O1]

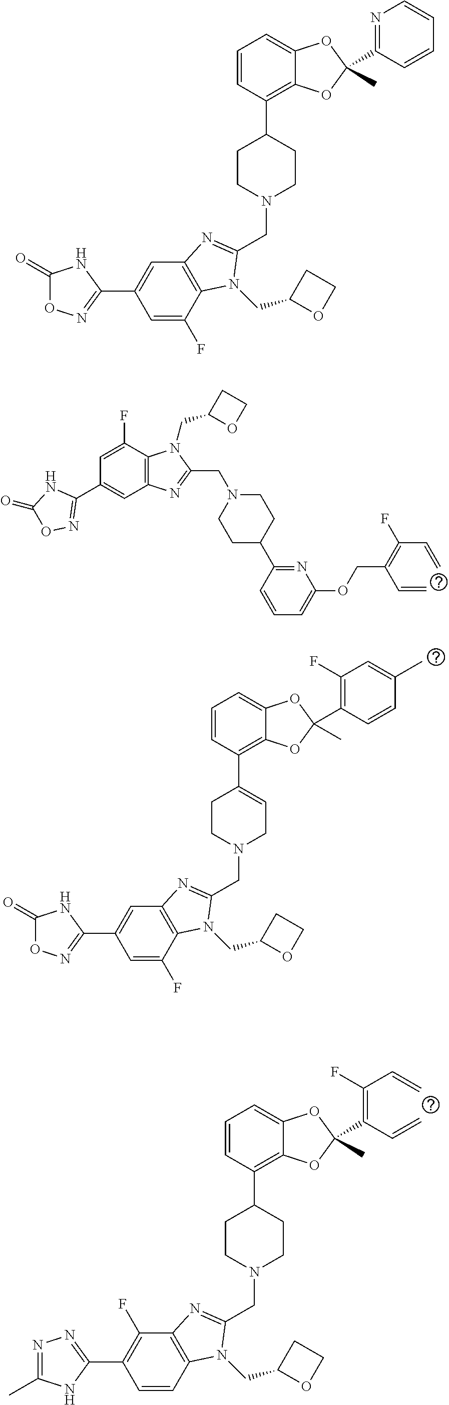

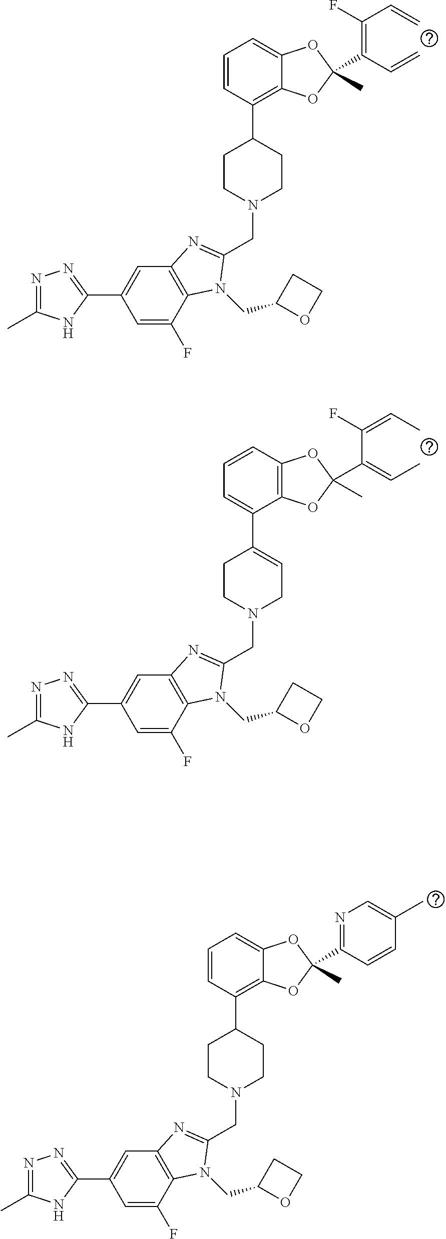

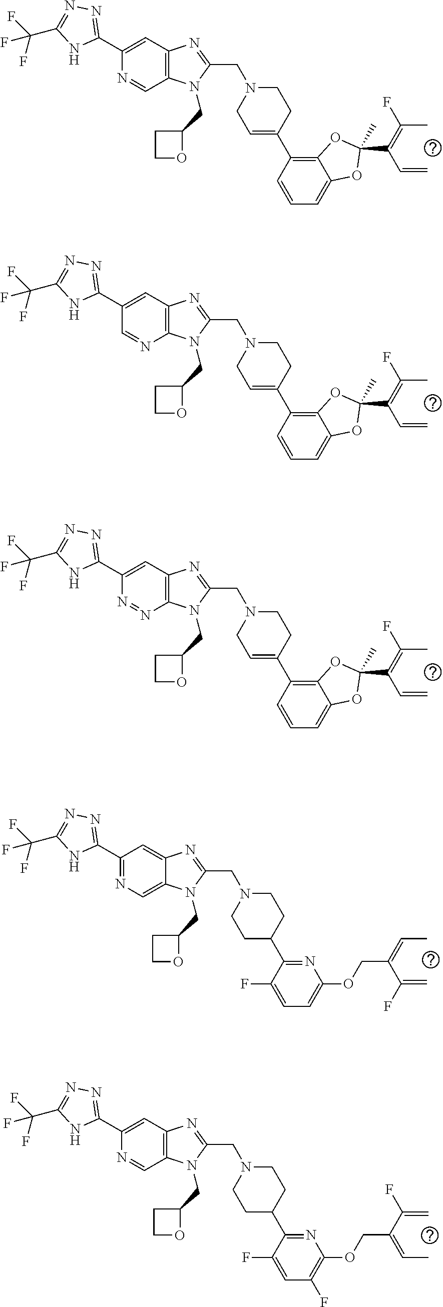 [SMILES: C=C/C(=C(\C)F)[C@@]1(C)Oc2cccc(C3=CCN(Cc4nc5cc(-c6nnc(C(F)(F)F)[nH]6)ncc5n4C[C@@H]4CCO4)CC3)c2O1 ; C=C/C(=C(\C)F)[C@@]1(C)Oc2cccc(C3=CCN(Cc4nc5cc(-c6nnc(C(F)(F)F)[nH]6)cnc5n4C[C@@H]4CCO4)CC3)c2O1 ; C=C/C(=C(\C)F)[C@@]1(C)Oc2cccc(C3=CCN(Cc4nc5cc(-c6nnc(C(F)(F)F)[nH]6)nnc5n4C[C@@H]4CCO4)CC3)c2O1 ; C=C(F)/C(=C\C)COc1ccc(F)c(C2CCN(Cc3nc4cc(-c5nnc(C(F)(F)F)[nH]5)ncc4n3C[C@@H]3CCO3)CC2)n1 ; C=C(F)/C(=C\C)COc1nc(C2CCN(Cc3nc4cc(-c5nnc(C(F)(F)F)[nH]5)ncc4n3C[C@@H]3CCO3)CC2)c(F)cc1F]

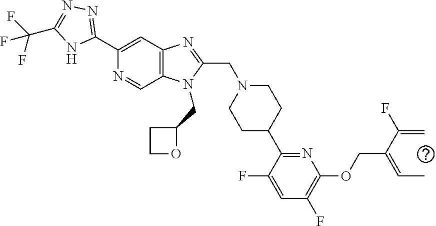 [SMILES: C=C(F)/C(=C\C)COc1nc(C2CCN(Cc3nc4cc(-c5nnc(C(F)(F)F)[nH]5)ncc4n3C[C@@H]3CCO3)CC2)c(F)cc1F]

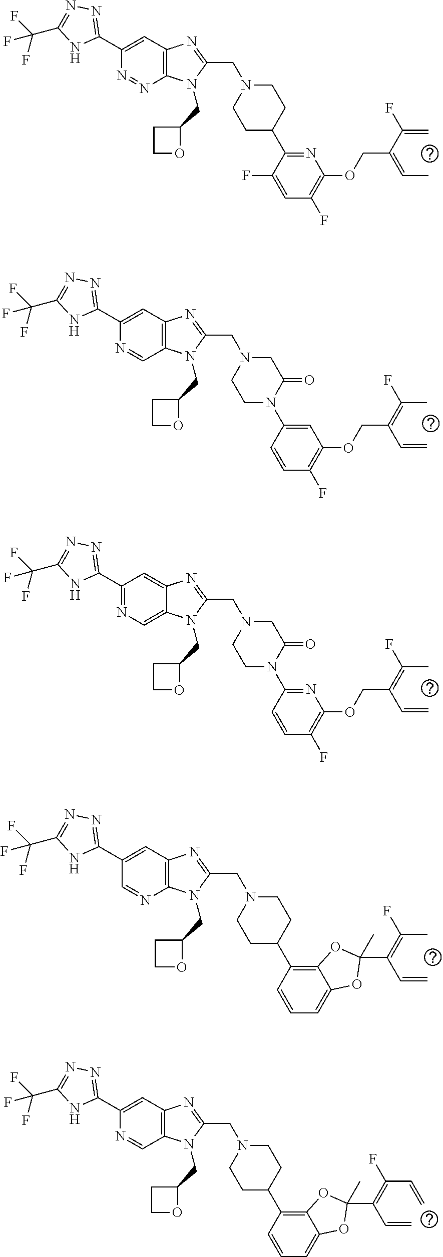 [SMILES: C=C(F)/C(=C\C)COc1nc(C2CCN(Cc3nc4cc(-c5nnc(C(F)(F)F)[nH]5)nnc4n3C[C@@H]3CCO3)CC2)c(F)cc1F ; C=C/C(COc1cc(N2CCN(Cc3nc4cc(-c5nnc(C(F)(F)F)[nH]5)ncc4n3C[C@@H]3CCO3)CC2=O)ccc1F)=C(\C)F ; C=C/C(COc1nc(N2CCN(Cc3nc4cc(-c5nnc(C(F)(F)F)[nH]5)ncc4n3C[C@@H]3CCO3)CC2=O)ccc1F)=C(\C)F ; C=C/C(=C(\C)F)C1(C)Oc2cccc(C3CCN(Cc4nc5cc(-c6nnc(C(F)(F)F)[nH]6)cnc5n4C[C@@H]4CCO4)CC3)c2O1 ; C=C/C(F)=C(\C=C)C1(C)Oc2cccc(C3CCN(Cc4nc5cc(-c6nnc(C(F)(F)F)[nH]6)ncc5n4C[C@@H]4CCO4)CC3)c2O1]

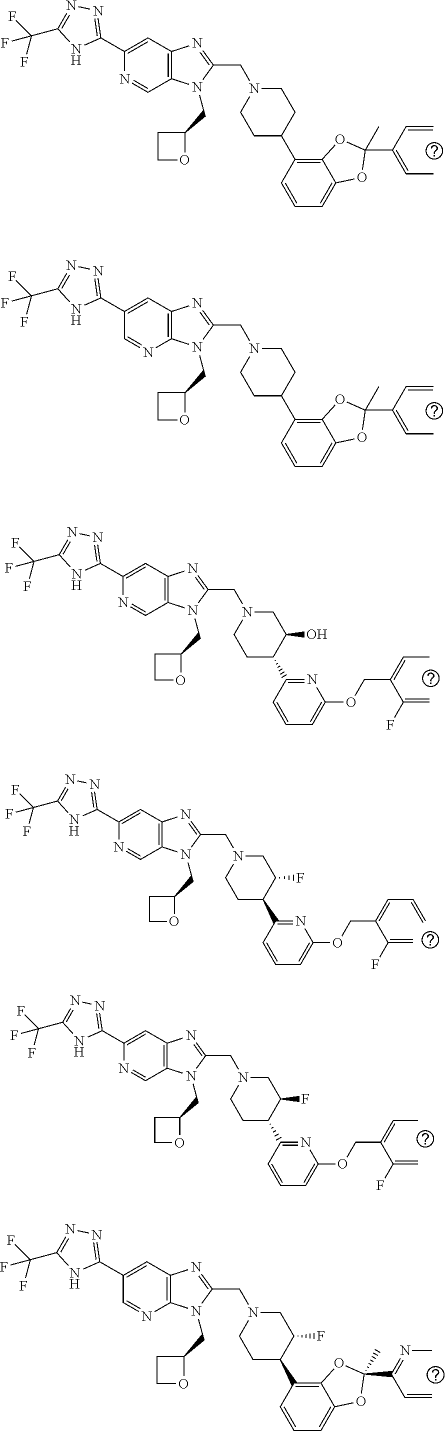

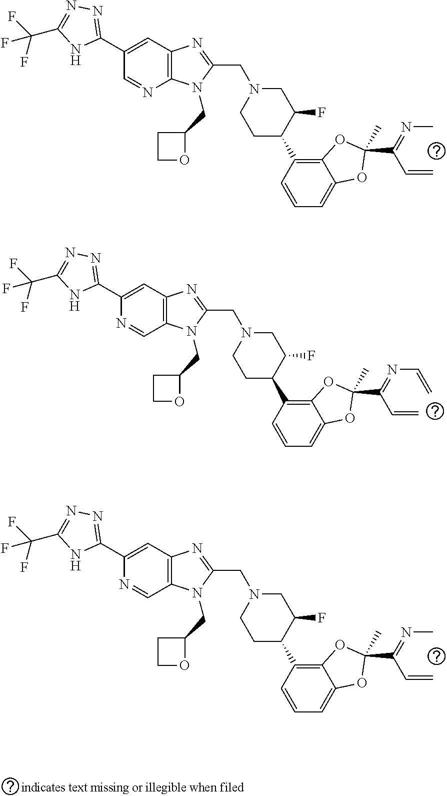 [SMILES: C=C/C(=N\C)[C@@]1(C)Oc2cccc([C@@H]3CCN(Cc4nc5cc(-c6nnc(C(F)(F)F)[nH]6)cnc5n4C[C@@H]4CCO4)C[C@H]3F)c2O1 ; C=C/N=C(\C=C)[C@@]1(C)Oc2cccc([C@H]3CCN(Cc4nc5cc(-c6nnc(C(F)(F)F)[nH]6)ncc5n4C[C@@H]4CCO4)C[C@@H]3F)c2O1 ; C=C/C(=N\C)[C@@]1(C)Oc2cccc([C@@H]3CCN(Cc4nc5cc(-c6nnc(C(F)(F)F)[nH]6)ncc5n4C[C@@H]4CCO4)C[C@H]3F)c2O1]

Examples of GLP-1 receptor agonists also include those disclosed in WO2023/179542, which is incorporated by reference as if fully set forth herein, falling within the general structure:

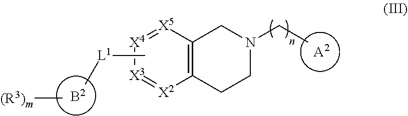

or a pharmaceutically acceptable salt or solvate thereof, wherein:
ring A is:

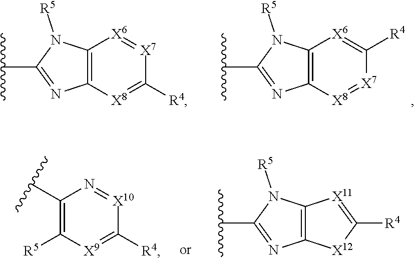

ring B is C₃₋₁₀cycloalkyl, heterocyclyl, aryl, or heteroaryl; one of X², X³, X⁴, and X⁵ is C covalently bonded to ring B via L¹; and the remaining of X², X³, X⁴, and X⁵ are each independently N or CR⁶; provided that no more than two of X², X³, X⁴, and X⁵ are N;
X⁶, X⁷ and X⁸ are each independently N or CR^{5A};
X⁹ and X¹⁰ are each independently N or CR^{6A};
X¹¹ is N or CR^{6A} and X¹² is S, O, or NR⁹;
n is 1, 2, or 3;
m is 0, 1, 2, 3, 4, or 5;
each R³ is independently halo, cyano, nitro, oxo, —OR¹⁰, —SR¹⁰, —N(R¹⁰)₂, —C(O)R¹⁰, —C(O)OR¹⁰, —OC(O)R¹⁰, —OC(O)OR¹⁰, —C(O)N(R¹⁰)₂, —NR¹⁰C(O)R¹⁰, —OC(O)N(R¹⁰)₂, —NR¹⁰C(O)OR¹⁰, —NR¹⁰C(O)N(R¹⁰)₂, —S(O)R¹⁰, —S(O)₂R¹⁰, —S(O)N(R¹⁰)₂, —S(O)₂N(R¹⁰)₂, —NR¹⁰S(O)R¹⁰, —NR¹⁰S(O)₂R¹⁰, —NR¹⁰S(O)N(R¹⁰)₂, —NR¹⁰S(O)₂N(R¹⁰)₂, C₁₋₉alkyl, C₂₋₉alkenyl, C₂₋₉alkynyl, C₃₋₁₀cycloalkyl, heterocyclyl, aryl, or heteroaryl; wherein each C₁₋₉alkyl, C₂₋₉alkenyl, C₂₋₉alkynyl, C₃₋₁₀cycloalkyl, heterocyclyl, aryl, or heteroaryl of R³ is independently optionally substituted with one to five Z²;
R⁴ is —C(O)OR⁹, —C(O)N(R⁹)₂, —C(O)N(R⁹)S(O)₂R⁹, —NR⁹C(O)R⁹, 5- to 10-membered heterocyclyl or 5- to 10-membered heteroaryl, wherein the 5- to 10-membered heteroaryl or 5- to 10-membered heterocyclyl is optionally substituted with 1-4 R¹²;
R⁵ is C₁₋₉alkyl optionally substituted with —O—(C₁₋₉alkyl), —S—(C₁₋₉alkyl), —S(O)₂—(C₁₋₉alkyl), C₃₋₆cycloalkyl, 3- to 6-membered heterocyclyl, phenyl, or 5- to 6-membered heteroaryl; wherein each C₁₋₉alkyl, —O—(C₁₋₉alkyl), —S—(C₁₋₉alkyl), —S(O)₂—(C₁₋₉alkyl), C₃₋₆cycloalkyl, 3- to 6-membered heterocyclyl, phenyl, or 5- to 6-membered heteroaryl of R⁵ is further optionally substituted with one to five Z²;
L¹ is a bond, C₁₋₉alkylene, C₂₋₉alkenylene, C₂₋₉alkynylene, —O—C₁₋₉alkylene, —NR¹⁰—C₁₋₉alkylene, —C(O)NR¹⁰—C₁₋₉alkylene, —NR¹⁰C(O)—C₁₋₉ alkylene, 3- to 6-membered heterocyclylene, —O—, —S—, —S(O)—, —S(O)₂—, —NR¹⁰—, —C(O)NR¹⁰—, —NR¹⁰C(O)—, —C(O)—, —OC(O)—, —C(O)O—, —NR¹⁰S(O)—, —S(O)NR¹⁰—, —NR¹⁰S(O)NR¹⁰—, —NR¹⁰S(O)₂NR¹⁰—, —NR¹⁰C(O)NR¹⁰—, —OC(O)NR¹⁰—, or —NR¹⁰C(O)O—; wherein each C₁₋₉ alkylene, C₂₋₉alkenylene, C₂₋₉alkynylene, —O—C₁₋₉alkylene, —NR¹⁰—C₁₋₉ alkylene, —C(O)NR¹⁰—C₁₋₉alkylene, —NR¹⁰C(O)—C₁₋₉alkylene, or 3- to 6-membered heterocyclylene of L¹ is independently optionally substituted with one to five Z²;
each R⁶ is independently hydrogen, halo, cyano, nitro, oxo, —OR¹⁰, —SR¹⁰, —N(R¹⁰)₂, —C(O)R¹⁰, —C(O)OR¹⁰, —OC(O)R¹⁰, —OC(O)OR¹⁰, —C(O)N(R¹⁰)₂, —NR¹⁰C(O)R¹⁰, —OC(O)N(R¹⁰)₂, —NR¹⁰C(O)OR¹⁰, —NR¹⁰C(O)N(R¹⁰)₂, —S(O)R¹⁰, —S(O)₂R¹⁰, —S(O)N(R¹⁰)₂, —S(O)₂N(R¹⁰)₂, —NR¹S(O)R¹⁰, —NR¹OS(O)₂R¹⁰, —NR¹S(O)N(R¹⁰)₂, —NR¹⁰S(O)₂N(R¹⁰)₂, C₁₋₉alkyl, C₂₋₉alkenyl, C₂₋₉alkynyl, C₃₋₁₀cycloalkyl, heterocyclyl, aryl, or heteroaryl; wherein each C₁₋₉alkyl, C₂₋₉alkenyl, C₂₋₉alkynyl, C₃₋₁₀cycloalkyl, heterocyclyl, aryl, or heteroaryl of R⁶ is independently optionally substituted with one to five Z²;
each R^{5A} is independently hydrogen, halo, cyano, nitro, oxo, —OH, —SH, —NH₂, —NH—C₁₋₆alkyl, —N(C₁₋₆alkyl)₂, —S—C₁₋₆alkyl, C₁₋₆alkoxy, C₁₋₆ alkyl, C₂₋₆alkenyl, or C₂₋₆alkynyl; wherein each —NH—C₁₋₆alkyl, —N(C₁₋₆ alkyl)₂, —S—C₁₋₆alkyl, C₁₋₆alkoxy, C₁₋₆alkyl, C₂₋₉alkenyl, or C₂₋₉alkynyl of R^{5A} is independently optionally substituted with one to five substituents independently selected from halo, hydroxy, and cyano;
each R^{6A} is independently hydrogen, halo, cyano, nitro, oxo, —OH, —SH, —NH₂, —NH—C₁₋₆alkyl, —N(C₁₋₆alkyl)₂, —S—C₁₋₆alkyl, C₁₋₆alkoxy, C₁₋₆alkyl, C₂₋₉alkenyl, or C₂₋₉alkynyl; wherein each —NH—C₁₋₆alkyl, —N(C₁₋₆ alkyl)₂, —S—C₁₋₆alkyl, C₁₋₆alkoxy, C₁₋₆alkyl, C₂₋₆alkenyl, or C₂₋₆alkynyl of R^{6A} is independently optionally substituted with one to five substituents independently selected from halo, hydroxy, and cyano;
each R⁹ is independently hydrogen, C₁₋₉alkyl, C₂₋₉alkenyl, C₂₋₉alkynyl, C₃₋₁₀cycloalkyl, heterocyclyl, aryl, heteroaryl; wherein each C₁₋₉alkyl, C₂₋₉alkenyl, C₂₋₉alkynyl, C₃₋₁₀cycloalkyl, heterocyclyl, aryl, or heteroaryl of R⁹ is independently optionally substituted with one to five R¹¹;
each R¹⁰ is independently hydrogen, C₁₋₉alkyl, C₂₋₉alkenyl, C₂₋₉alkynyl, C₃₋₁₀cycloalkyl, heterocyclyl, aryl, heteroaryl, —C(O)R²⁰, —C(O)OR²⁰, —C(O)N(R²⁰)₂, —S(O)R 20, —S(O)₂R²⁰, —S(O)N(R²⁰)₂, or —S(O)₂N(R²⁰)₂; wherein each C₁₋₉alkyl, C₂₋₉alkenyl, C₂₋₉alkynyl, C₃₋₁₀cycloalkyl, heterocyclyl, aryl, or heteroaryl of R¹⁰ is independently optionally substituted with one to five Z^{1a};
each R¹¹ is independently oxo, cyano, halo, hydroxy, C₁₋₉alkyl, C₁₋₉ alkoxy, C₁₋₉haloalkyl, C₁₋₉haloalkoxy, C₃₋₉cycloalkyl, —C₁₋₉alkyl-C(O)OR¹², —C(O)OR¹², —C(O)N(R¹²)₂, —SR¹², or —S(O)₂R¹²;
each R¹² is independently hydrogen, C₁₋₉alkyl, C₂₋₉alkenyl, C₂₋₉alkynyl, C₃₋₁₀cycloalkyl, heterocyclyl, aryl, or heteroaryl; wherein each C₁₋₉ alkyl, C₂₋₉alkenyl, C₂₋₉alkynyl, C₃₋₁₀cycloalkyl, heterocyclyl, aryl, or heteroaryl of R¹² is independently optionally substituted with one to five Z^{1a};
each Z² is independently halo, cyano, nitro, oxo, C₁₋₉alkyl, C₂₋₉alkenyl, C₂₋₉alkynyl, C₃₋₁₀cycloalkyl, heterocyclyl, aryl, heteroaryl, -L²-C₁₋₉alkyl, -L²-C₂₋₉alkenyl, -L²-C₂₋₉alkynyl, -L²-C₃₋₁₀cycloalkyl, -L²-heterocyclyl, -L²-aryl, or -L²-heteroaryl; wherein each C₁₋₉alkyl, C₂₋₉alkenyl, C₂₋₉alkynyl, C₃₋₁₀cycloalkyl, heterocyclyl, aryl, or heteroaryl of Z² is independently optionally substituted with one to five Z^{1a};
each L² is independently —O—, —S—, —NR²⁰—, —C(O)—, —C(O)O—, —OC(O)—, —OC(O)O—,
—C(O)NR²⁰—, —NR²⁰C(O)—, —OC(O)NR²⁰—, —NR²⁰C(O)O—, —NR²⁰C(O)NR²⁰—, —(O)—, —S(O)₂—, —S(O)NR²⁰—, —S(O)₂NR²⁰—, —NR²⁰S(O)—, —NR²⁰S(O)₂—, —NR²⁰S(O)NR²⁰—, or —NR²⁰S(O)₂NR²⁰—;
each R²⁰ is independently hydrogen, C₁₋₉alkyl, C₂₋₉alkenyl, C₂₋₉alkynyl, C₃₋₁₀cycloalkyl, heterocyclyl, aryl, or heteroaryl; wherein each C₁₋₉ alkyl, C₂₋₉alkenyl, C₂₋₉alkynyl, C₃₋₁₀cycloalkyl, heterocyclyl, aryl, or heteroaryl of R²⁰ is independently optionally substituted with one to five Z^{1a};
each Z^{1a} is independently halo, hydroxy, cyano, nitro, oxo, —SH, —NH₂, —NH—C₁₋₆alkyl, —N(C₁₋₆alkyl)₂, —S—C₁₋₆alkyl, C₁₋₆alkoxy, C₁₋₆alkyl, C₂₋₆alkenyl, C₂₋₆alkynyl, C₃₋₆cycloalkyl, heterocyclyl, aryl, or heteroaryl; wherein each C₁₋₆alkyl, C₂₋₆alkenyl, C₂₋₆alkynyl, C₃₋₆cycloalkyl, heterocyclyl, aryl, or heteroaryl of Z^{1a} is independently optionally substituted with one to five substituents selected from C₁₋₉alkyl, oxo, halo, hydroxy, and cyano.

Examples of compounds of the formula (III) include, but are not limited to, the following:

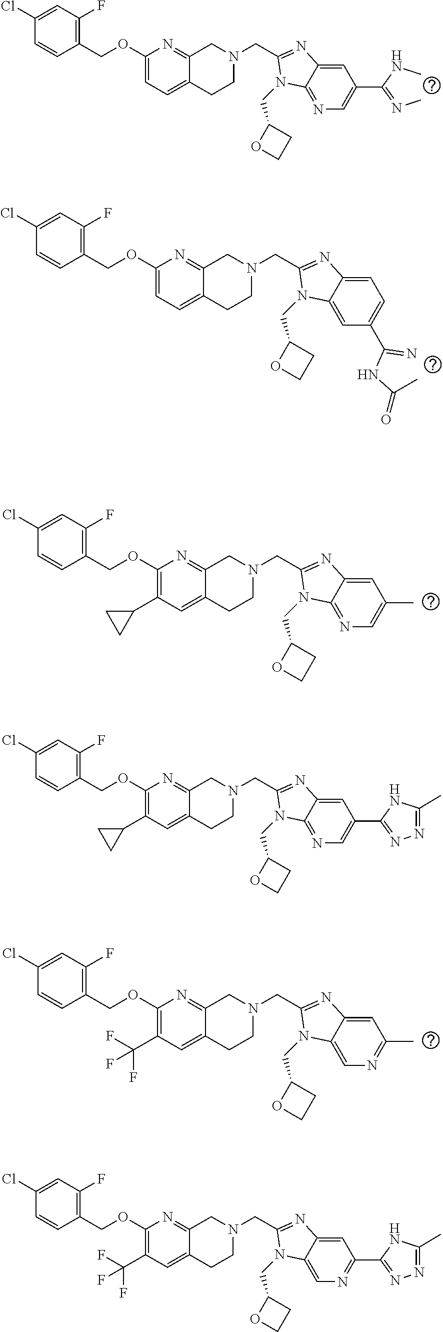 [SMILES: C/N=C(\NC)c1cnc2c(c1)nc(CN1CCc3ccc(OCc4ccc(Cl)cc4F)nc3C1)n2C[C@@H]1CCO1 ; CC(=O)NC(=N)c1ccc2nc(CN3CCc4ccc(OCc5ccc(Cl)cc5F)nc4C3)n(C[C@@H]3CCO3)c2c1 ; Cc1cnc2c(c1)nc(CN1CCc3cc(C4CC4)c(OCc4ccc(Cl)cc4F)nc3C1)n2C[C@@H]1CCO1 ; Cc1nnc(-c2cnc3c(c2)nc(CN2CCc4cc(C5CC5)c(OCc5ccc(Cl)cc5F)nc4C2)n3C[C@@H]2CCO2)[nH]1 ; Cc1cc2nc(CN3CCc4cc(C(F)(F)F)c(OCc5ccc(Cl)cc5F)nc4C3)n(C[C@@H]3CCO3)c2cn1 ; Cc1nnc(-c2cc3nc(CN4CCc5cc(C(F)(F)F)c(OCc6ccc(Cl)cc6F)nc5C4)n(C[C@@H]4CCO4)c3cn2)[nH]1]

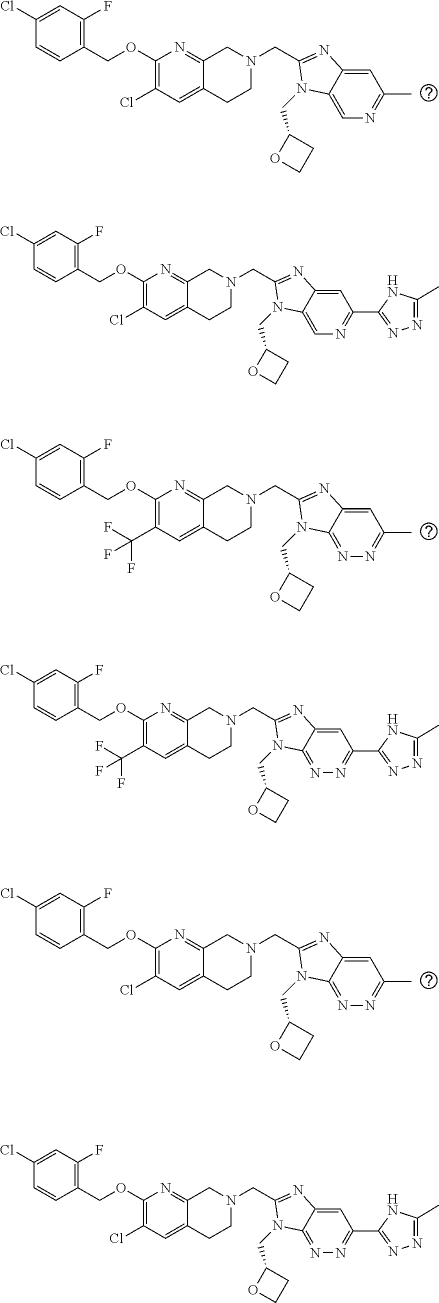 [SMILES: Cc1cc2nc(CN3CCc4cc(Cl)c(OCc5ccc(Cl)cc5F)nc4C3)n(C[C@@H]3CCO3)c2cn1 ; Cc1nnc(-c2cc3nc(CN4CCc5cc(Cl)c(OCc6ccc(Cl)cc6F)nc5C4)n(C[C@@H]4CCO4)c3cn2)[nH]1 ; Cc1cc2nc(CN3CCc4cc(C(F)(F)F)c(OCc5ccc(Cl)cc5F)nc4C3)n(C[C@@H]3CCO3)c2nn1 ; Cc1nnc(-c2cc3nc(CN4CCc5cc(C(F)(F)F)c(OCc6ccc(Cl)cc6F)nc5C4)n(C[C@@H]4CCO4)c3nn2)[nH]1 ; Cc1cc2nc(CN3CCc4cc(Cl)c(OCc5ccc(Cl)cc5F)nc4C3)n(C[C@@H]3CCO3)c2nn1 ; Cc1nnc(-c2cc3nc(CN4CCc5cc(Cl)c(OCc6ccc(Cl)cc6F)nc5C4)n(C[C@@H]4CCO4)c3nn2)[nH]1]

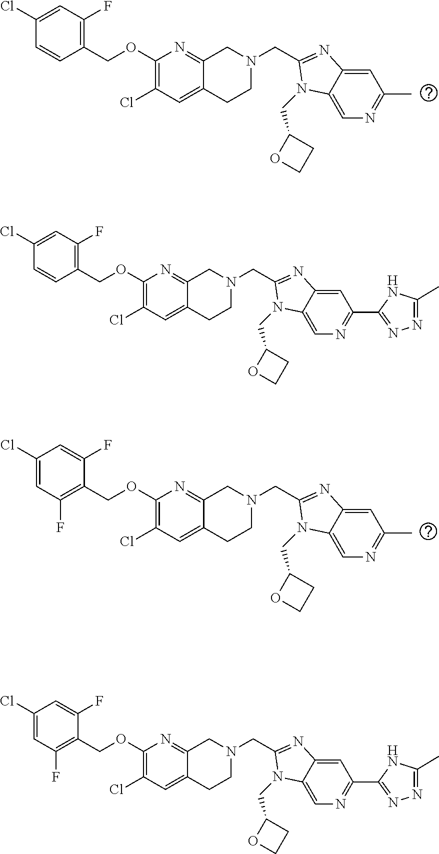 [SMILES: Cc1cc2nc(CN3CCc4cc(Cl)c(OCc5ccc(Cl)cc5F)nc4C3)n(C[C@@H]3CCO3)c2cn1 ; Cc1nnc(-c2cc3nc(CN4CCc5cc(Cl)c(OCc6ccc(Cl)cc6F)nc5C4)n(C[C@@H]4CCO4)c3cn2)[nH]1 ; Cc1cc2nc(CN3CCc4cc(Cl)c(OCc5c(F)cc(Cl)cc5F)nc4C3)n(C[C@@H]3CCO3)c2cn1 ; Cc1nnc(-c2cc3nc(CN4CCc5cc(Cl)c(OCc6c(F)cc(Cl)cc6F)nc5C4)n(C[C@@H]4CCO4)c3cn2)[nH]1]

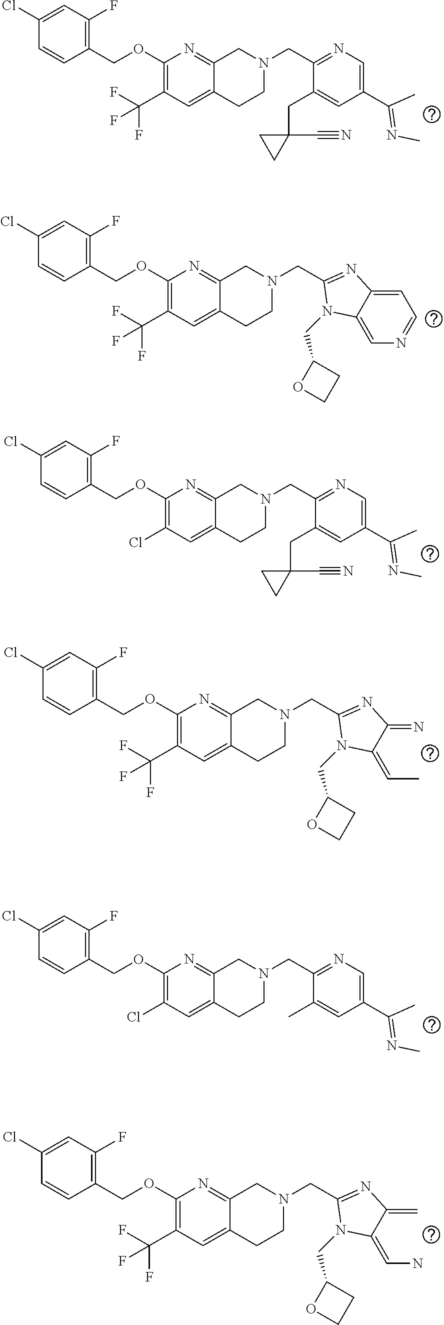 [SMILES: C/N=C(\C)c1cnc(CN2CCc3cc(C(F)(F)F)c(OCc4ccc(Cl)cc4F)nc3C2)c(CC2(C#N)CC2)c1 ; Fc1cc(Cl)ccc1COc1nc2c(cc1C(F)(F)F)CCN(Cc1nc3ccncc3n1C[C@@H]1CCO1)C2 ; C/N=C(\C)c1cnc(CN2CCc3cc(Cl)c(OCc4ccc(Cl)cc4F)nc3C2)c(CC2(C#N)CC2)c1 ; C/C=C1\C(=N)N=C(CN2CCc3cc(C(F)(F)F)c(OCc4ccc(Cl)cc4F)nc3C2)N1C[C@@H]1CCO1 ; C/N=C(\C)c1cnc(CN2CCc3cc(Cl)c(OCc4ccc(Cl)cc4F)nc3C2)c(C)c1 ; C=c1nc(CN2CCc3cc(C(F)(F)F)c(OCc4ccc(Cl)cc4F)nc3C2)n(C[C@@H]2CCO2)/c1=C/N]

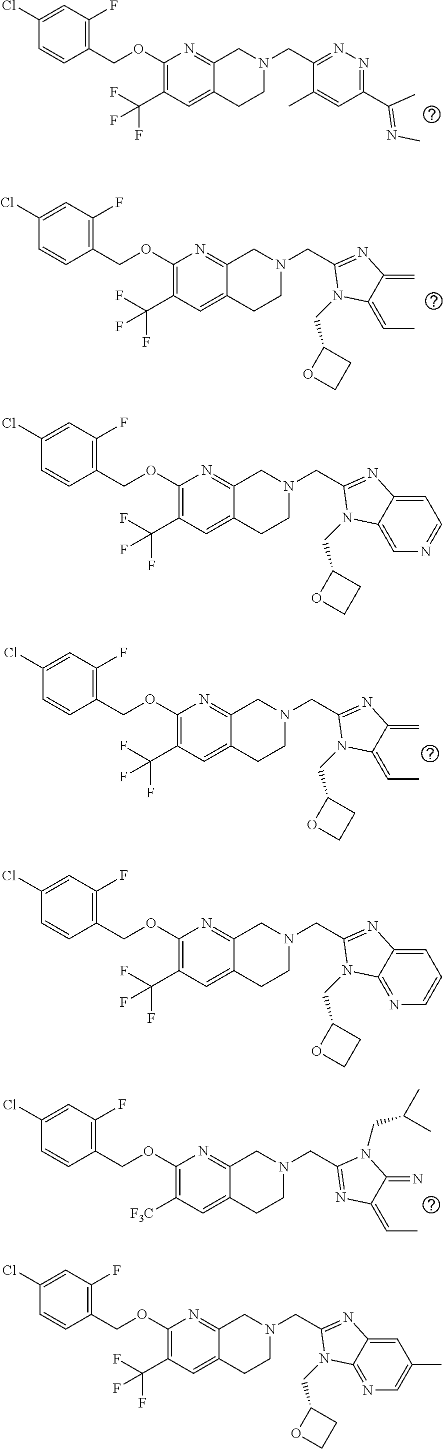

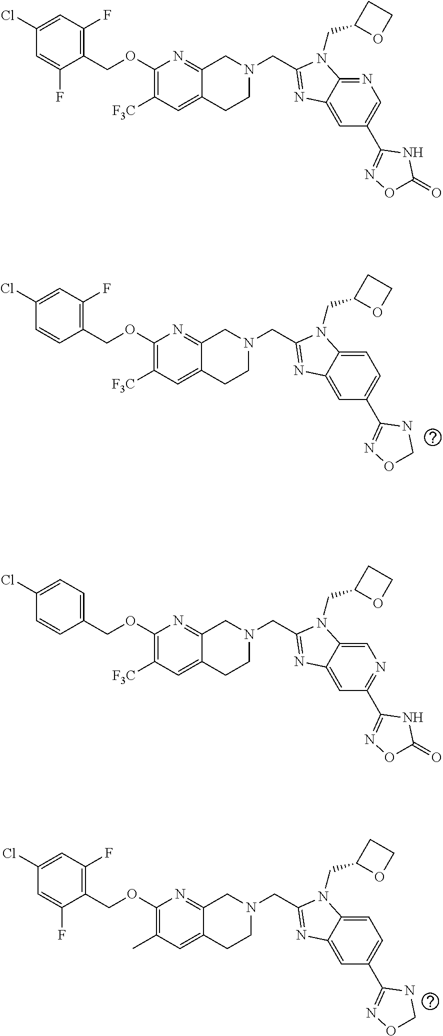 [SMILES: O=c1[nH]c(-c2cnc3c(c2)nc(CN2CCc4cc(C(F)(F)F)c(OCc5c(F)cc(Cl)cc5F)nc4C2)n3C[C@@H]2CCO2)no1 ; Fc1cc(Cl)ccc1COc1nc2c(cc1C(F)(F)F)CCN(Cc1nc3cc(C4=NOCN4)ccc3n1C[C@@H]1CCO1)C2 ; O=c1[nH]c(-c2cc3nc(CN4CCc5cc(C(F)(F)F)c(OCc6ccc(Cl)cc6)nc5C4)n(C[C@@H]4CCO4)c3cn2)no1 ; Cc1cc2c(nc1OCc1c(F)cc(Cl)cc1F)CN(Cc1nc3cc(C4=NOCN4)ccc3n1C[C@@H]1CCO1)CC2]

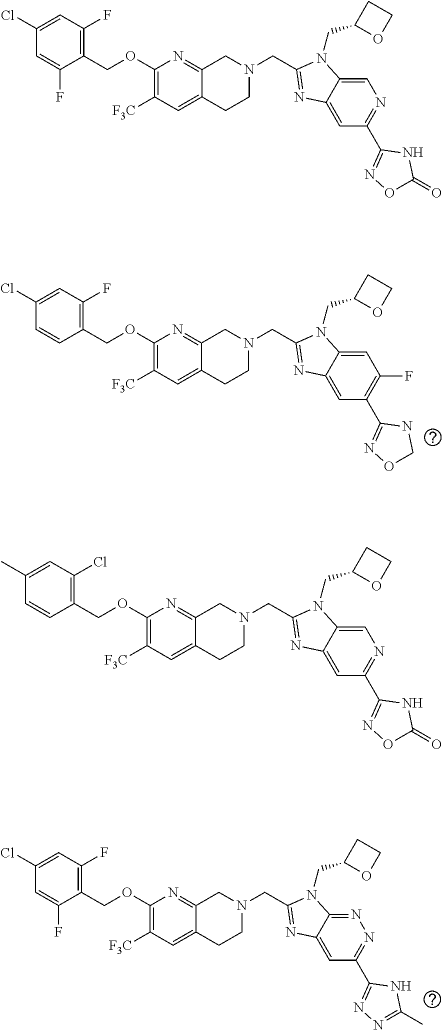 [SMILES: O=c1[nH]c(-c2cc3nc(CN4CCc5cc(C(F)(F)F)c(OCc6c(F)cc(Cl)cc6F)nc5C4)n(C[C@@H]4CCO4)c3cn2)no1 ; Fc1cc(Cl)ccc1COc1nc2c(cc1C(F)(F)F)CCN(Cc1nc3cc(C4=NOCN4)c(F)cc3n1C[C@@H]1CCO1)C2 ; Cc1ccc(COc2nc3c(cc2C(F)(F)F)CCN(Cc2nc4cc(-c5noc(=O)[nH]5)ncc4n2C[C@@H]2CCO2)C3)c(Cl)c1 ; Cc1nnc(-c2cc3nc(CN4CCc5cc(C(F)(F)F)c(OCc6c(F)cc(Cl)cc6F)nc5C4)n(C[C@@H]4CCO4)c3nn2)[nH]1]

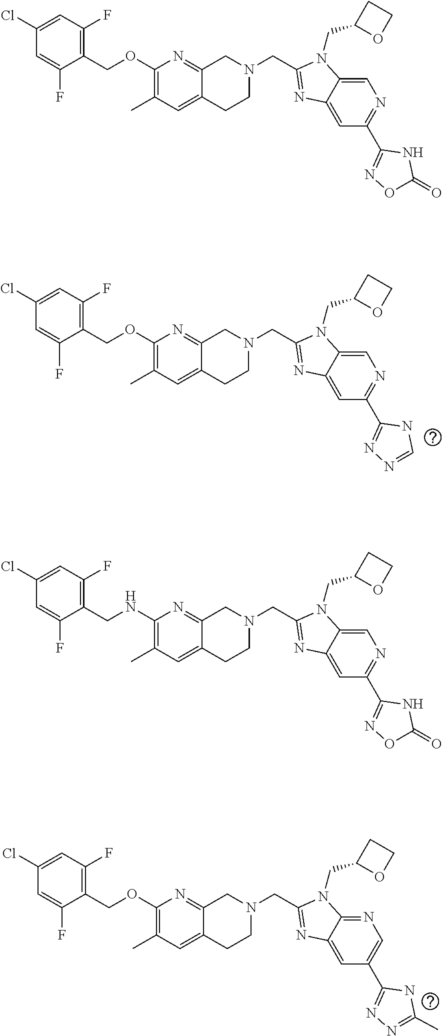 [SMILES: Cc1cc2c(nc1OCc1c(F)cc(Cl)cc1F)CN(Cc1nc3cc(-c4noc(=O)[nH]4)ncc3n1C[C@@H]1CCO1)CC2 ; Cc1cc2c(nc1OCc1c(F)cc(Cl)cc1F)CN(Cc1nc3cc(-c4nnc[nH]4)ncc3n1C[C@@H]1CCO1)CC2 ; Cc1cc2c(nc1NCc1c(F)cc(Cl)cc1F)CN(Cc1nc3cc(-c4noc(=O)[nH]4)ncc3n1C[C@@H]1CCO1)CC2 ; Cc1nnc(-c2cnc3c(c2)nc(CN2CCc4cc(C)c(OCc5c(F)cc(Cl)cc5F)nc4C2)n3C[C@@H]2CCO2)[nH]1]

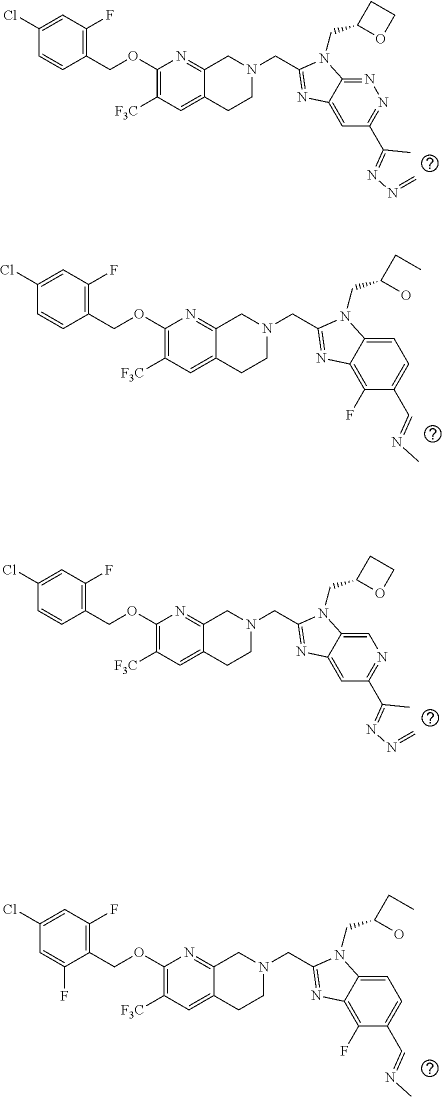 [SMILES: C=N/N=C(\C)c1cc2nc(CN3CCc4cc(C(F)(F)F)c(OCc5ccc(Cl)cc5F)nc4C3)n(C[C@@H]3CCO3)c2nn1 ; CC[C@H](O)Cn1c(CN2CCc3cc(C(F)(F)F)c(OCc4ccc(Cl)cc4F)nc3C2)nc2c(F)c(/C=N/C)ccc21 ; C=N/N=C(\C)c1cc2nc(CN3CCc4cc(C(F)(F)F)c(OCc5ccc(Cl)cc5F)nc4C3)n(C[C@@H]3CCO3)c2cn1 ; CC[C@H](O)Cn1c(CN2CCc3cc(C(F)(F)F)c(OCc4c(F)cc(Cl)cc4F)nc3C2)nc2c(F)c(/C=N/C)ccc21]

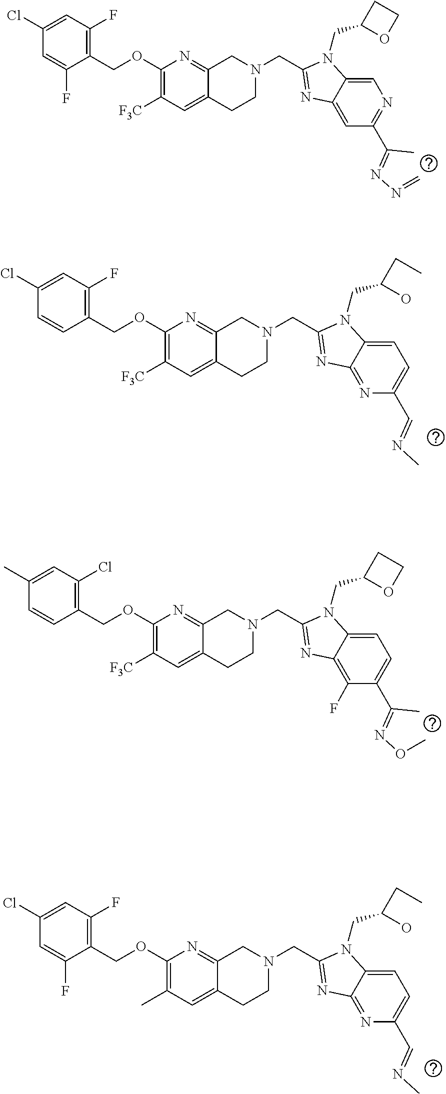

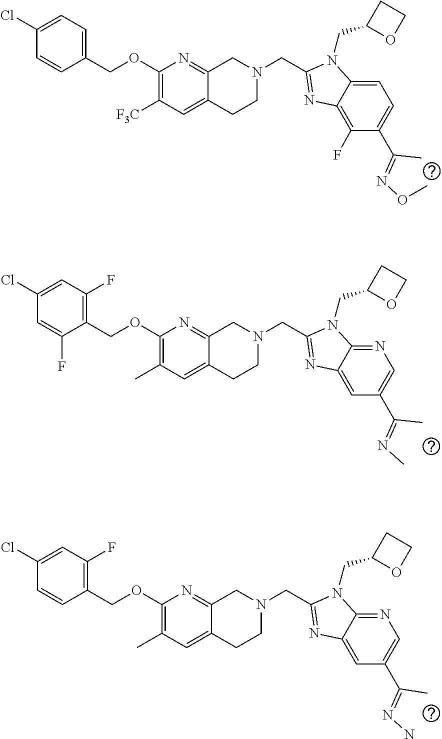 [SMILES: CO/N=C(\C)c1ccc2c(nc(CN3CCc4cc(C(F)(F)F)c(OCc5ccc(Cl)cc5)nc4C3)n2C[C@@H]2CCO2)c1F ; C/N=C(\C)c1cnc2c(c1)nc(CN1CCc3cc(C)c(OCc4c(F)cc(Cl)cc4F)nc3C1)n2C[C@@H]1CCO1 ; C/C(=N\N)c1cnc2c(c1)nc(CN1CCc3cc(C)c(OCc4ccc(Cl)cc4F)nc3C1)n2C[C@@H]1CCO1]

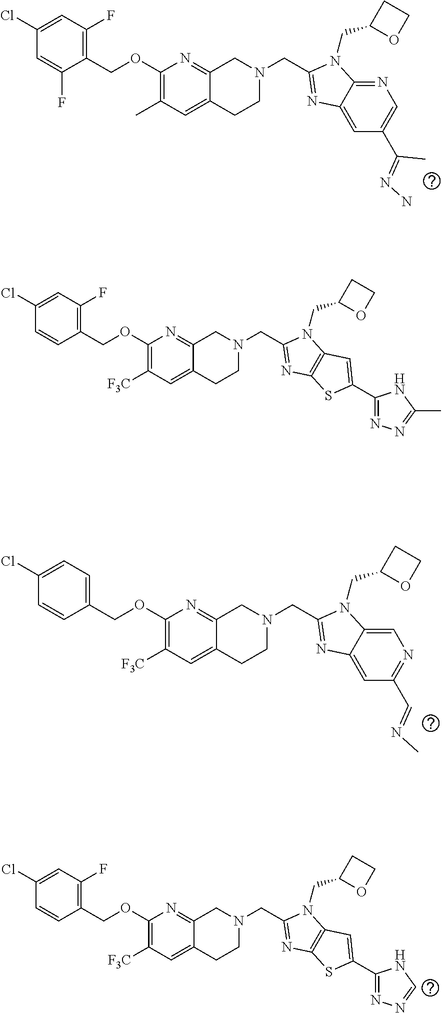 [SMILES: C/C(=N\N)c1cnc2c(c1)nc(CN1CCc3cc(C)c(OCc4c(F)cc(Cl)cc4F)nc3C1)n2C[C@@H]1CCO1 ; Cc1nnc(-c2cc3c(nc(CN4CCc5cc(C(F)(F)F)c(OCc6ccc(Cl)cc6F)nc5C4)n3C[C@@H]3CCO3)s2)[nH]1 ; C/N=C/c1cc2nc(CN3CCc4cc(C(F)(F)F)c(OCc5ccc(Cl)cc5)nc4C3)n(C[C@@H]3CCO3)c2cn1 ; Fc1cc(Cl)ccc1COc1nc2c(cc1C(F)(F)F)CCN(Cc1nc3sc(-c4nnc[nH]4)cc3n1C[C@@H]1CCO1)C2]

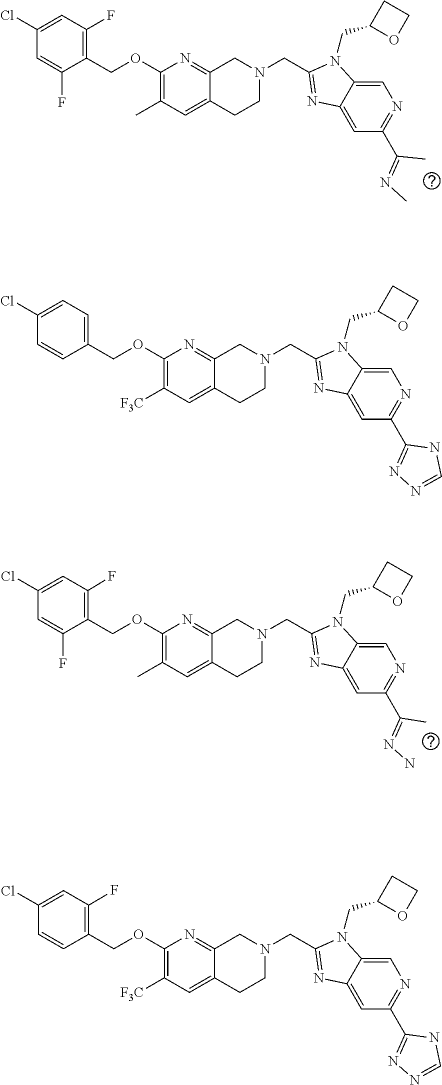 [SMILES: C/N=C(\C)c1cc2nc(CN3CCc4cc(C)c(OCc5c(F)cc(Cl)cc5F)nc4C3)n(C[C@@H]3CCO3)c2cn1 ; FC(F)(F)c1cc2c(nc1OCc1ccc(Cl)cc1)CN(Cc1nc3cc(-c4nnc[nH]4)ncc3n1C[C@@H]1CCO1)CC2 ; C/C(=N\N)c1cc2nc(CN3CCc4cc(C)c(OCc5c(F)cc(Cl)cc5F)nc4C3)n(C[C@@H]3CCO3)c2cn1 ; Fc1cc(Cl)ccc1COc1nc2c(cc1C(F)(F)F)CCN(Cc1nc3cc(-c4nnc[nH]4)ncc3n1C[C@@H]1CCO1)C2]

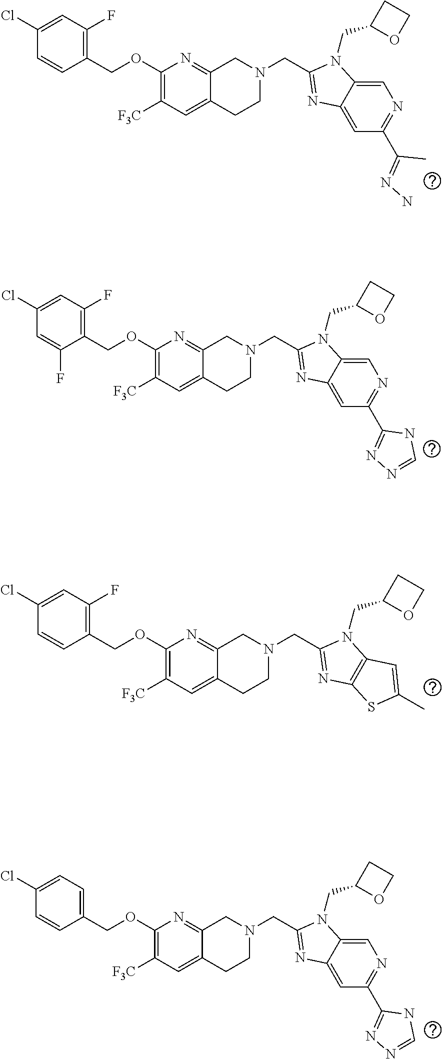 [SMILES: C/C(=N\N)c1cc2nc(CN3CCc4cc(C(F)(F)F)c(OCc5ccc(Cl)cc5F)nc4C3)n(C[C@@H]3CCO3)c2cn1 ; Fc1cc(Cl)cc(F)c1COc1nc2c(cc1C(F)(F)F)CCN(Cc1nc3cc(-c4nnc[nH]4)ncc3n1C[C@@H]1CCO1)C2 ; Cc1cc2c(nc(CN3CCc4cc(C(F)(F)F)c(OCc5ccc(Cl)cc5F)nc4C3)n2C[C@@H]2CCO2)s1 ; FC(F)(F)c1cc2c(nc1OCc1ccc(Cl)cc1)CN(Cc1nc3cc(-c4nnc[nH]4)ncc3n1C[C@@H]1CCO1)CC2]

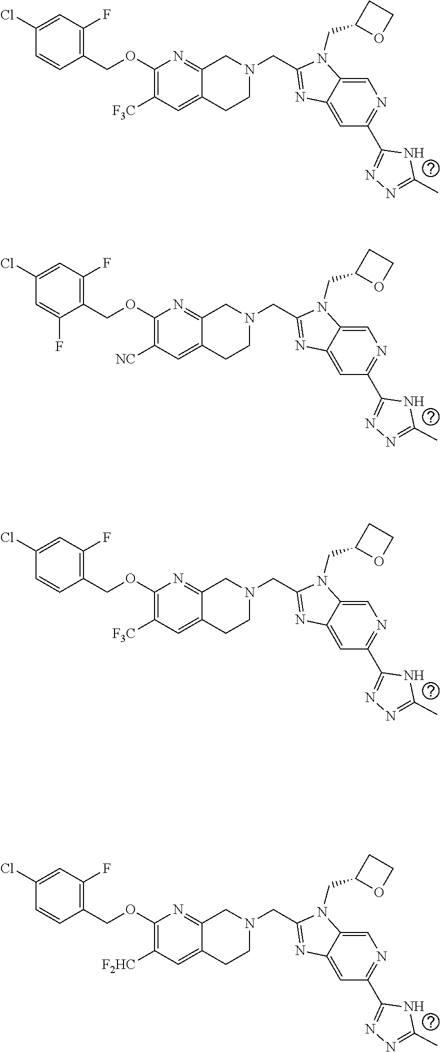 [SMILES: Cc1nnc(-c2cc3nc(CN4CCc5cc(C(F)(F)F)c(OCc6ccc(Cl)cc6F)nc5C4)n(C[C@@H]4CCO4)c3cn2)[nH]1 ; Cc1nnc(-c2cc3nc(CN4CCc5cc(C#N)c(OCc6c(F)cc(Cl)cc6F)nc5C4)n(C[C@@H]4CCO4)c3cn2)[nH]1 ; Cc1nnc(-c2cc3nc(CN4CCc5cc(C(F)(F)F)c(OCc6ccc(Cl)cc6F)nc5C4)n(C[C@@H]4CCO4)c3cn2)[nH]1 ; Cc1nnc(-c2cc3nc(CN4CCc5cc(C(F)F)c(OCc6ccc(Cl)cc6F)nc5C4)n(C[C@@H]4CCO4)c3cn2)[nH]1]

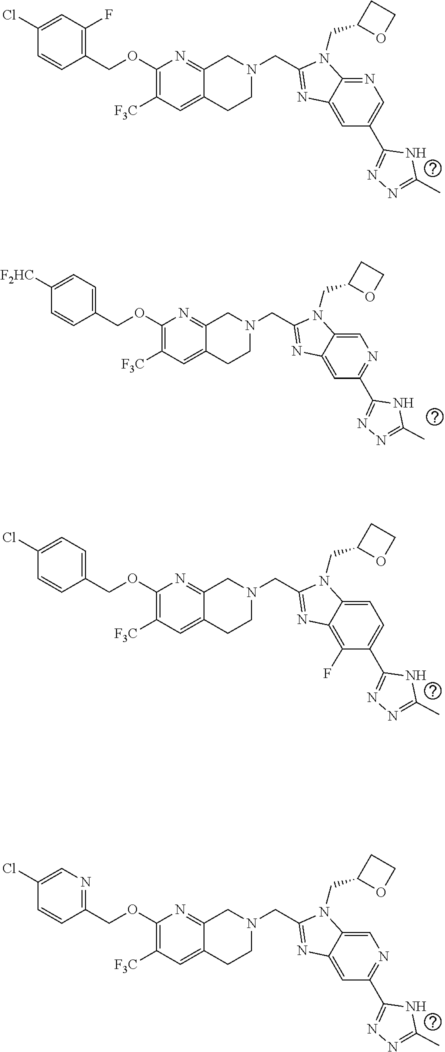 [SMILES: Cc1nnc(-c2cnc3c(c2)nc(CN2CCc4cc(C(F)(F)F)c(OCc5ccc(Cl)cc5F)nc4C2)n3C[C@@H]2CCO2)[nH]1 ; Cc1nnc(-c2cc3nc(CN4CCc5cc(C(F)(F)F)c(OCc6ccc(C(F)F)cc6)nc5C4)n(C[C@@H]4CCO4)c3cn2)[nH]1 ; Cc1nnc(-c2ccc3c(nc(CN4CCc5cc(C(F)(F)F)c(OCc6ccc(Cl)cc6)nc5C4)n3C[C@@H]3CCO3)c2F)[nH]1 ; Cc1nnc(-c2cc3nc(CN4CCc5cc(C(F)(F)F)c(OCc6ccc(Cl)cn6)nc5C4)n(C[C@@H]4CCO4)c3cn2)[nH]1]

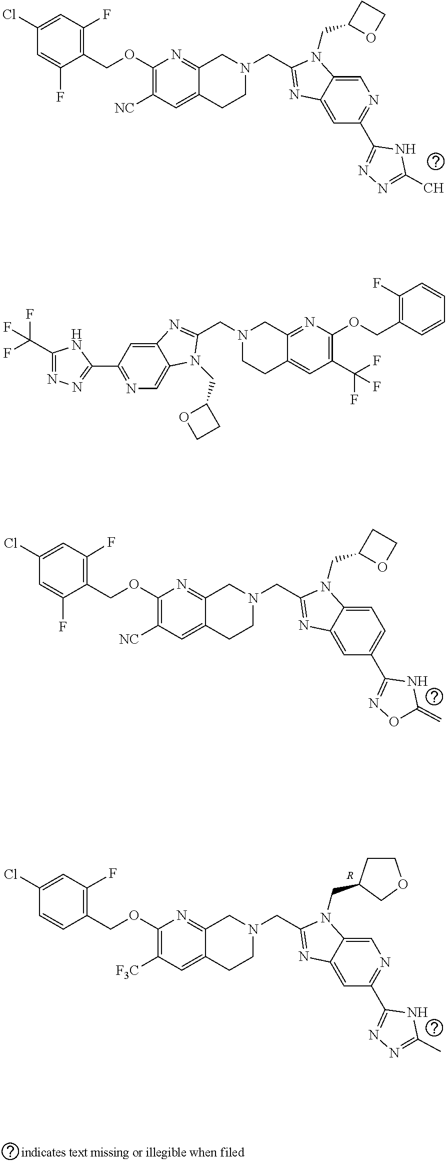

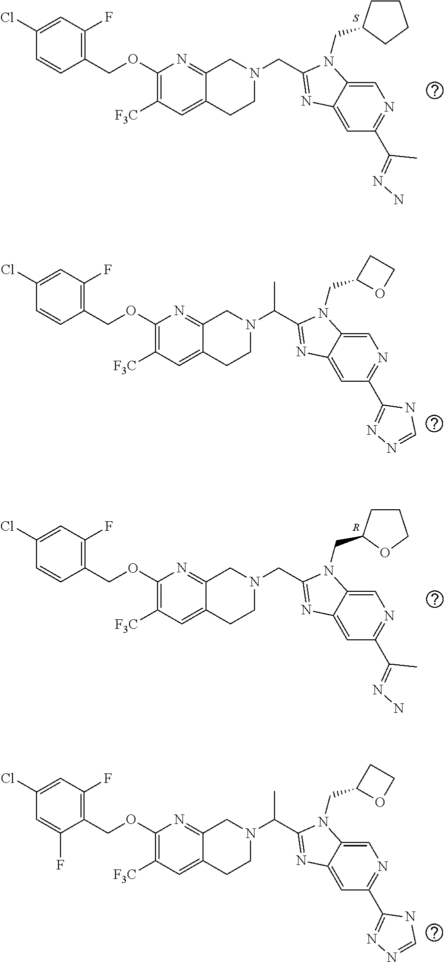

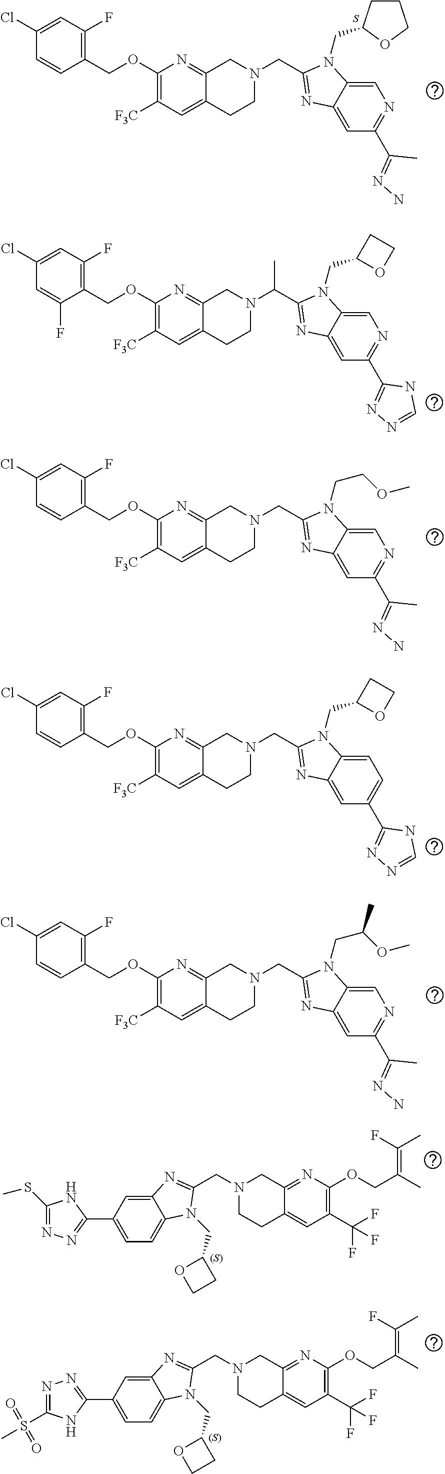

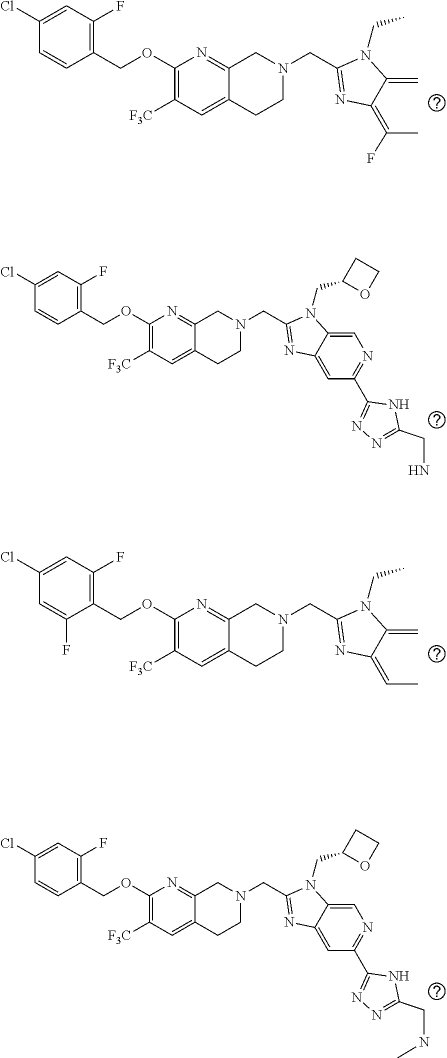

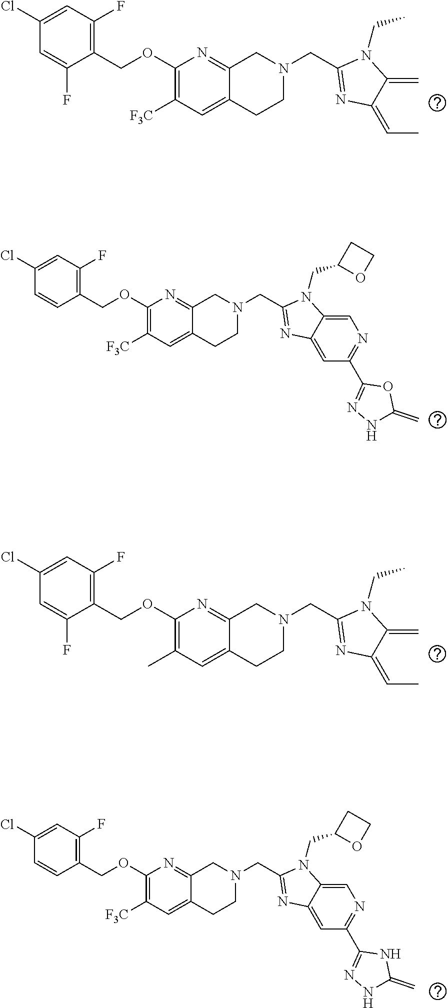

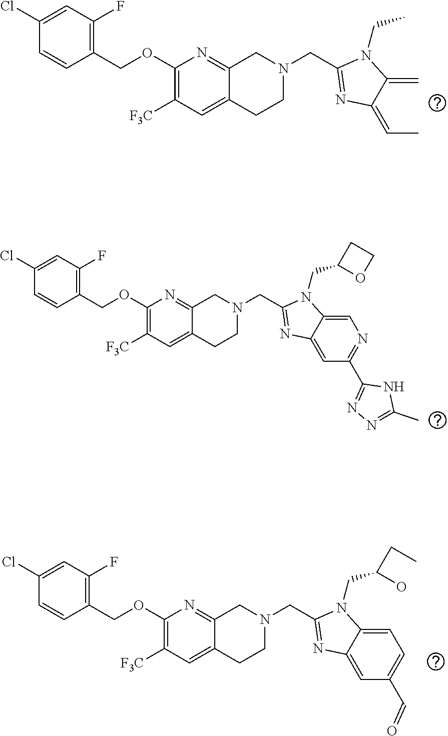

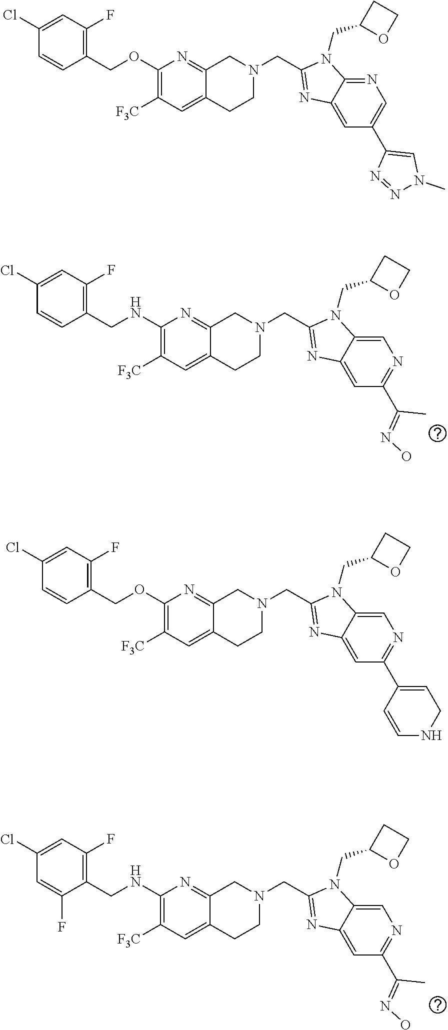 [SMILES: Cn1cc(-c2cnc3c(c2)nc(CN2CCc4cc(C(F)(F)F)c(OCc5ccc(Cl)cc5F)nc4C2)n3C[C@@H]2CCO2)nn1 ; C/C(=N\O)c1cc2nc(CN3CCc4cc(C(F)(F)F)c(NCc5ccc(Cl)cc5F)nc4C3)n(C[C@@H]3CCO3)c2cn1 ; Fc1cc(Cl)ccc1COc1nc2c(cc1C(F)(F)F)CCN(Cc1nc3cc(C4=CCNC=C4)ncc3n1C[C@@H]1CCO1)C2 ; C/C(=N\O)c1cc2nc(CN3CCc4cc(C(F)(F)F)c(NCc5c(F)cc(Cl)cc5F)nc4C3)n(C[C@@H]3CCO3)c2cn1]

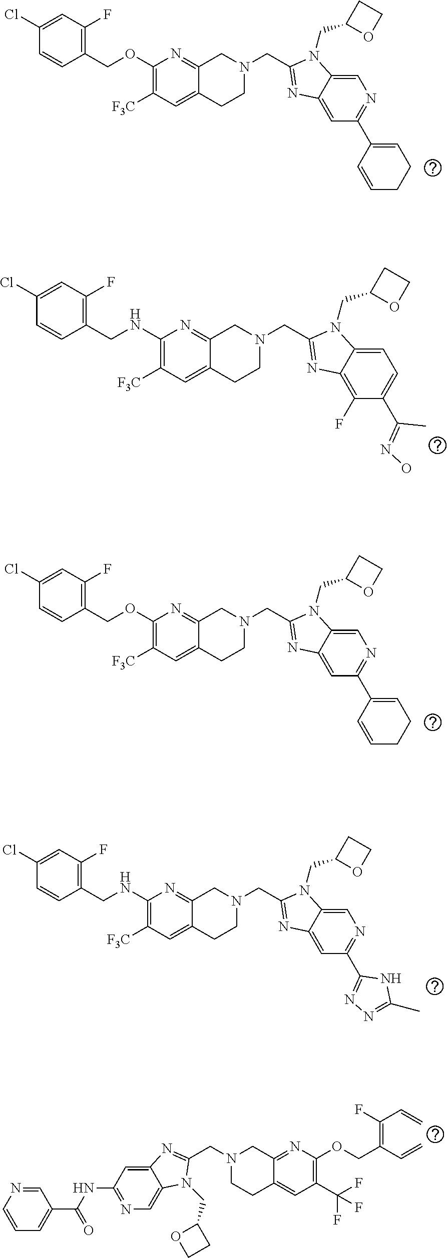 [SMILES: Fc1cc(Cl)ccc1COc1nc2c(cc1C(F)(F)F)CCN(Cc1nc3cc(C4=CCCC=C4)ncc3n1C[C@@H]1CCO1)C2 ; C/C(=N\O)c1ccc2c(nc(CN3CCc4cc(C(F)(F)F)c(NCc5ccc(Cl)cc5F)nc4C3)n2C[C@@H]2CCO2)c1F ; Fc1cc(Cl)ccc1COc1nc2c(cc1C(F)(F)F)CCN(Cc1nc3cc(C4=CCCC=C4)ncc3n1C[C@@H]1CCO1)C2 ; Cc1nnc(-c2cc3nc(CN4CCc5cc(C(F)(F)F)c(NCc6ccc(Cl)cc6F)nc5C4)n(C[C@@H]4CCO4)c3cn2)[nH]1 ; C=C/C(F)=C(\C=C)COc1nc2c(cc1C(F)(F)F)CCN(Cc1nc3cc(NC(=O)c4cccnc4)ncc3n1C[C@@H]1CCO1)C2]

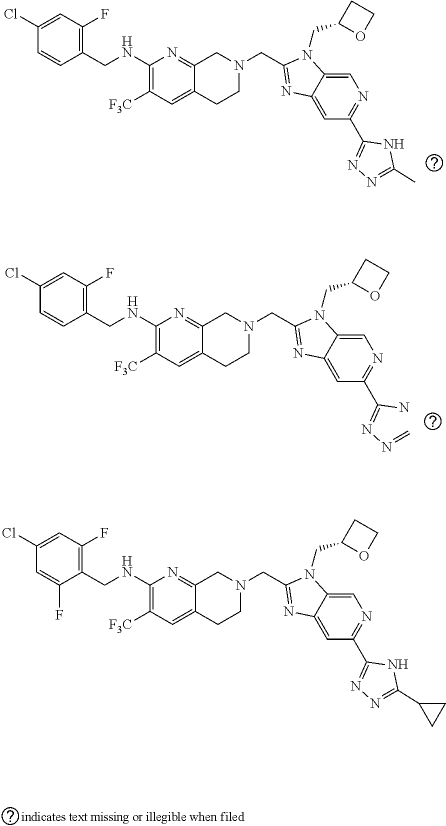 [SMILES: Cc1nnc(-c2cc3nc(CN4CCc5cc(C(F)(F)F)c(NCc6ccc(Cl)cc6F)nc5C4)n(C[C@@H]4CCO4)c3cn2)[nH]1 ; C=N/N=C(\N)c1cc2nc(CN3CCc4cc(C(F)(F)F)c(NCc5ccc(Cl)cc5F)nc4C3)n(C[C@@H]3CCO3)c2cn1 ; Fc1cc(Cl)cc(F)c1CNc1nc2c(cc1C(F)(F)F)CCN(Cc1nc3cc(-c4nnc(C5CC5)[nH]4)ncc3n1C[C@@H]1CCO1)C2]

Additional GLP-1 receptor agonists contemplated herein include, but are not limited to any and all GLP-1 receptor agonists disclosed in: WO23169456; WO23151575; WO23151574; WO23138684; WO22079639; US2023241178; U.S. Pat. No. 9,585,959; US2012021979; US2011105389; U.S. Pat. No. 9,161,988; US2006241100; U.S. Pat. No. 7,151,097; WO06131730; U.S. Pat. No. 7,141,669; EP1622903; U.S. Pat. Nos. 7,268,133; 7,145,012; 7,176,210; 7,247,628; 7,129,239; and US2004077650, each of which is incorporated by reference as if fully set forth herein.

Although the free-base form of the GLP-1 agonists can be used in the methods of the present invention, it is preferred to prepare and use a pharmaceutically acceptable salt thereof. Thus, the compounds used in the methods of this invention form pharmaceutically acceptable acid and base addition salts with a wide variety of inorganic and, preferably, organic acids and include the physiologically acceptable salts which are often used in pharmaceutical chemistry. Such salts are also part of this invention. Typical inorganic acids used to form such salts include hydrochloric, hydrobromic, hydroiodic, nitric, sulfuric, phosphoric, hypophosphoric, and the like. Salts derived from organic acids, such as aliphatic mono and dicarboxylic acids, phenyl substituted alkanoic acids, hydroxyalkanoic and hydroxyalkandioic acids, aromatic acids, aliphatic and aromatic sulfonic acids, may also be used. Such pharmaceutically acceptable salts thus include acetate, phenylacetate, trifluoroacetate, acrylate, ascorbate, benzoate, chlorobenzoate, dinitrobenzoate, hydroxybenzoate, methoxybenzoate, methylbenzoate, o-acetoxybenzoate, naphthalene-2-benzoate, bromide, isobutyrate, phenylbutyrate, p-hydroxybutyrate, butyne-1,4-dioate, hexyne-1,4-dioate, caprate, caprylate, chloride, cinnamate, citrate, formate, fumarate, glycollate, heptanoate, hippurate, lactate, malate, maleate, hydroxymaleate, malonate, mandelate, mesylate, nicotinate, isonicotinate, nitrate, oxalate, phthalate, terephthalate, phosphate, monohydrogenphosphate, propriolate, propionate, phenyl-propionate, salicylate, sebacate, succinate, suberate, sulfate, bisulfate, pyrosulate, sulfite, bisulfite, sulfonate, benzenesulfonate, p-bromophenylsulfonate, chlorobenzenesulfonate, ethanesulfonate, 2-hydroxyethanesulfonate, methanesulfonate, ethanesulfonate, 2-hydroxyethanesulfonate, methanesulfonate, naphthalene-1-sulfonate, naphthalene-2-sulfonate, p-toluenesulfonate, xylenesulfonate, tartarate, and the like.

The pharmaceutically acceptable acid addition salts are typically formed by reacting the free base with an equimolar or excess amount of acid. The reactants are generally combined in a mutual solvent such as diethyl ether or benzene. The salt normally precipitates out of solution within about one hour to 10 days and can be isolated by filtration or the solvent can be removed by conventional means.

The pharmaceutically acceptable salts generally have enhanced solubility characteristics compared to the compound from which they are derived, and thus are often more amenable to formulation as liquids or emulsions.

The compounds useful in the present method can be administered by a variety of routes including oral, rectal, transdermal, subcutaneous, intravenous, intramuscular, and intranasal. These compounds preferably are formulated prior to administration, the selection of which will be decided by the attending physician.

Typically, one or more agonists, or pharmaceutically acceptable salts or esters thereof, is combined with a pharmaceutically acceptable carrier, diluent or excipient to form a pharmaceutical formulation, or unit dosage form.

The total active ingredients in such formulations comprise from 0.1% to 99.9% by weight of the formulation. By “pharmaceutically acceptable” it is meant that the carrier, diluent, excipients, and/or salt must be compatible with the other ingredients of the formulation, and not deleterious to the recipient thereof.

Pharmaceutical formulations containing the agonist or agonists can be prepared by procedures known in the art using well-known and readily available ingredients. For example, the agonist can be formulated with common excipients, diluents, or carriers, and formed into tablets, capsules, suspensions, powders, and the like. Examples of excipients, diluents, and carriers that are suitable for such formulations include the following fillers and extenders such as starch, sugars, mannitol, and silicic derivatives binding agents such as carboxymethyl cellulose and other cellulose derivatives, alginate, gelatin, and polyvinyl-pyrrolidone; moisturizing agents such as glycerol; disintegrating agents such as calcium carbonate and sodium bicarbonate; agents for retarding dissolution such as paraffin; resorption accelerators such as quaternary ammonium compounds; surface active agents such as cetyl alcohol, glycerol monostearate; adsorptive carriers such as kaolin and bentonite; and lubricants such as talc, calcium and magnesium stearate and solid polyethylene glycols.

The compounds also can be formulated as tablets or in capsules or as elixirs or solutions for convenient oral administration or as solutions appropriate for parenteral administration, for example, by intramuscular, subcutaneous or intravenous routes.

Additionally, the compounds are well suited to formulation as sustained or controlled release dosage forms. The formulations can be so constituted that they release the active ingredient only or preferably in a particular physiological location, optionally over a period of time. The coatings, envelopes, and protective matrices may be made, for example, from polymeric substances such as collagen or silicone, or from waxes. The compounds can also be delivered via patches for transdermal delivery, s.c. implants, infusion pumps or release from implanted depot sustained release dosage forms.

As used herein, the term “effective amount” means an amount of compound which is capable of inhibiting at least one of the symptoms of the ICDs herein described. The specific dose of a compound administered according to this invention will, of course, be determined by the particular circumstances surrounding the case including, for example, the compound administered, the route of administration, the condition of the patient, and the severity of the symptoms being treated. A typical daily dose will contain a nontoxic dosage level of from about 0.25 mg to about 500 mg/day of an opioid receptor agonist of the present invention. Preferred daily doses generally will be from about 1 mg to about 300 mg/day.

The terms “substituted,” “substituent,” and “functional group” refer to a group that can be or is substituted onto a molecule or onto another group (e.g., on an aryl or an alkyl group). Examples of substituents include, but are not limited to, a halogen (e.g., F, Cl, Br, and I), OR, OC(O)N(R)₂, CN, NO, NO₂, ONO₂, azido, CF₃, OCF₃, R, O (oxo), S (thiono), C(O), S(O), methylenedioxy, ethylenedioxy, N(R)₂, SR, SOR, SO₂R, SO₂N(R)₂, SO₃R, —(CH₂)₀₋₂P(O)(OR)₂, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)C(O)OR, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, or C(═NOR)R wherein each R can be, independently, hydrogen, alkyl, acyl, cycloalkyl, aryl, aralkyl, heterocyclyl, heteroaryl, or heteroarylalkyl, wherein any alkyl, acyl, cycloalkyl, aryl, aralkyl, heterocyclyl, heteroaryl, or heteroarylalkyl or two R groups bonded to a nitrogen atom or to adjacent nitrogen atoms can together with the nitrogen atom or atoms form a heterocyclyl, which can be mono- or independently multi-substituted.

The term “alkyl” as used herein refers to substituted or unsubstituted straight chain and branched mono- or divalent alkyl groups and cycloalkyl groups having from 1 to 40 carbon atoms (C₁-C₄₀), 1 to about 20 carbon atoms (C₁-C₂₀), 1 to 12 carbons (C₁-C₁₂), 1 to 8 carbon atoms (C₁-C₈), or, in some embodiments, from 1 to 6 carbon atoms (C₁-C₆). Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and ante-isoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “alkenyl” as used herein refers to substituted or unsubstituted straight chain and branched mono- or divalent alkenyl groups and cycloalkenyl groups having at least one double bond and having from 1 to 40 carbon atoms (C₁-C₄₀), 1 to about 20 carbon atoms (C₁-C₂₀), 1 to 12 carbons (C₁-C₁₂), 1 to 8 carbon atoms (C₁-C₈), or, in some embodiments, from 1 to 6 carbon atoms (C₁-C₆). Examples of straight chain alkenyl groups include those with from 1 to 8 carbon atoms such as —CH═CH—, —CH═CHCH₃, and —CH₂CH═CHCH₂— groups, wherein the double bonds can have an E- or Z-configuration. And when there are multiple bonds, each double bond can, independently, have an E- or a Z-configuration. Examples of branched alkenyl groups include, but are not limited to, —CH═C(CH₃)— and CH₂C═CH(CH₃) groups. Representative substituted alkenyl groups can be substituted one or more times with any of the groups listed herein, for example, amino, hydroxy, cyano, carboxy, nitro, thio, alkoxy, and halogen groups.

The term “cycloalkyl” as used herein refers to substituted or unsubstituted cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. Cycloalkyl groups can have any number of carbon atoms, e.g., 3 to 8 carbon atoms (C₃-C₈), 3 to 6 carbon atoms (C₃-C₆), and 4 to 8 carbon atoms (C₄-C₈). Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like.

The term “cycloalkylalkyl” as used herein refers to substituted or unsubstituted alkyl groups as defined herein in which a hydrogen or carbon bond of an alkyl group as defined herein is replaced with a bond to a cycloalkyl group as defined herein. Representative cycloalkylalkyl groups include, but are not limited to, cyclopentylalkyl.

The term “alkylcycloalkyl” as used herein refers to substituted or unsubstituted cycloalkyl groups as defined herein in which a hydrogen of a cycloalkyl group as defined herein is replaced with a bond to an alkyl group as defined herein. Representative alkylcycloalkyl groups include, but are not limited to, alkylcyclopropyl.

The term “acyl” as used herein refers to a group containing a carbonyl moiety wherein the group is bonded via the carbonyl carbon atom. The carbonyl carbon atom is also bonded to another carbon atom, which can be part of a substituted or unsubstituted alkyl, aryl, aralkyl cycloalkyl, cycloalkylalkyl, heterocyclyl, heterocyclylalkyl, heteroaryl, heteroarylalkyl group or the like. In the special case wherein the carbonyl carbon atom is bonded to a hydrogen, the group is a “formyl” group, an acyl group as the term is defined herein. An acyl group can include 0 to about 12-40, 6-10, 1-5 or 2-5 additional carbon atoms bonded to the carbonyl group. An acryloyl group is an example of an acyl group. An acyl group can also include heteroatoms within the meaning here. A nicotinoyl group (pyridyl-3-carbonyl) is an example of an acyl group within the meaning herein. Other examples include acetyl, benzoyl, phenylacetyl, pyridylacetyl, cinnamoyl, and acryloyl groups and the like. When the group containing the carbon atom that is bonded to the carbonyl carbon atom contains a halogen, the group is termed a “haloacyl” group. An example is a trifluoroacetyl group.

The term “heterocyclylcarbonyl” is an example of an acyl group that is bonded to a substituted or unsubstituted heterocyclyl group, as the term “heterocyclyl” is defined herein. An example of a heterocyclylcarbonyl group is a prolyl group, wherein the prolyl group can be a D- or an L-prolyl group.

The term “aryl” as used herein refers to substituted or unsubstituted cyclic aromatic hydrocarbons that do not contain heteroatoms in the ring. Thus, aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons (C₆-C₁₄) or from 6 to 10 carbon atoms (C₆-C₁₀) in the ring portions of the groups. Aryl groups can be unsubstituted or substituted, as defined herein. “Aryl” and the phrase “aryl group” includes fused ring species including those that include fused aromatic and non-aromatic groups. Accordingly, “aryl” and the phrase “aryl group” include groups of the formula:

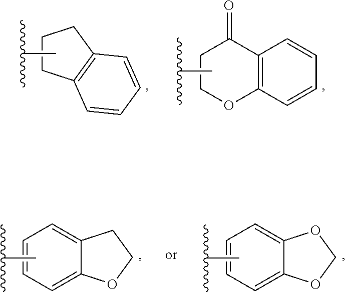

each of which can be substituted or unsubstituted, such as hydroxy substituted.

Representative substituted aryl groups can be mono-substituted or substituted more than once, such as, but not limited to, 2-, 3-, 4-, 5-, or 6-substituted phenyl or 2-8 substituted naphthyl groups, which can be substituted with carbon or non-carbon groups such as those listed herein.

The terms “aralkyl” and “arylalkyl” refer to alkyl groups as defined herein in which a hydrogen or carbon bond of an alkyl group is replaced with a bond to an aryl group as defined herein. Representative aralkyl groups include benzyl and phenylethyl groups and fused (cycloalkylaryl)alkyl groups such as 4-ethyl-indanyl. Aralkenyl groups are alkenyl groups as defined herein in which a hydrogen or carbon bond of an alkyl group is replaced with a bond to an aryl group as defined herein.

The term “heterocyclyl” or “heterocyclo” refers to substituted or unsubstituted aromatic and non-aromatic ring compounds containing 3 or more ring members, of which one or more (e.g., 1, 2 or 3) is a heteroatom such as, but not limited to, N, O, and S. Thus, a heterocyclyl can be a cycloheteroalkyl or a heteroaryl or, if polycyclic, any combination thereof. In some embodiments, heterocyclyl groups include 3 to about 20 ring members, whereas other such groups have 3 to about 15 ring members. In some embodiments, heterocyclyl groups include heterocyclyl groups that include 3 to 8 carbon atoms (C₃-C₅), 3 to 6 carbon atoms (C₃-C₆), 3 to 5 carbon atoms (C₃-C₅) or 6 to 8 carbon atoms (C₆-C₅). A heterocyclyl group designated as a C₂-heterocyclyl can be a 5-ring with two carbon atoms and three heteroatoms, a 6-ring with two carbon atoms and the heteroatoms and so forth. Likewise, a C₄-heterocyclyl can be a 5-ring with one heteroatom, a 6-ring with two heteroatoms, and so forth. The number of carbon atoms plus the number of heteroatoms equals the total number of ring atoms. A heterocyclyl ring can also include one or more double bonds, such as in the group 3,6-dihydro-2H-pyran and 3,4-dihydro-2H-pyran, having the formula:

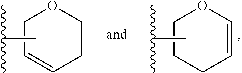

respectively, each of which can be substituted.

A heteroaryl ring is an embodiment of a heterocyclyl group. The phrase “heterocyclyl group” includes fused ring species including those that include fused aromatic and non-aromatic groups. Representative heterocyclyl groups include, but are not limited to tetrahydro-2H-thiopyran-1,1-dioxide, having the formula:

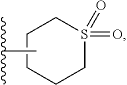

which can be substituted, 4a,5,6,7-tetrahydro-4H-pyrrolo[1,2-d][1,3,4]oxadiazinyl, having the formula:

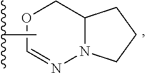

which can be substituted, pyrrolidinyl, pyrrolidinone (e.g., pyrrolidin-2-one), azetidinyl, piperidynyl, piperazinyl, morpholinyl, chromanyl, indolinonyl, isoindolinonyl, furanyl, pyrrolidinyl, pyridinyl, pyrazinyl, pyrimidinyl, triazinyl, thiophenyl, tetrahydrofuranyl, pyrrolyl, oxazolyl, oxadiazolyl, imidazolyl, imidazo[1,2-a]pyridinyl, having the formula:

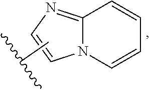

which can be substituted, triazyolyl, tetrazolyl, benzoxazolinyl, thiazolyl, benzthiazolinyl, and benzimidazolinyl groups. Examples of indolinonyl groups include groups having the general formula:

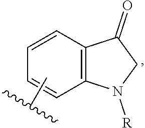

wherein R is as defined herein.

Examples of isoindolinonyl groups include groups having the general formula:

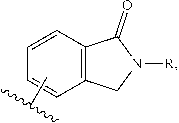

wherein R is as defined herein.

Examples of benzoxazolinyl groups include groups having the general formula:

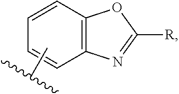

wherein R is as defined herein.

Examples of benzthiazolinyl groups include groups having the general formula:

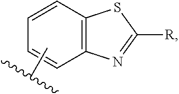

wherein R is as defined herein.

In some embodiments, the group R in benzoxazolinyl and benzthiazolinyl groups is an N(R)₂ group. In some embodiments, each R is hydrogen or alkyl, wherein the alkyl group is substituted or unsubstituted. In some embodiments, the alkyl group is substituted with a heterocyclyl group (e.g., with a pyrrolidinyl group).

The term “heterocyclylalkyl” refers to alkyl groups as defined herein in which a hydrogen or carbon bond of an alkyl group as defined herein is replaced with a bond to a heterocyclyl group as defined herein. Representative heterocyclylalkyl groups include, but are not limited to, furan-2-yl methyl, furan-3-yl methyl, pyridine-3-yl methyl, tetrahydrofuran-2-yl methyl, and indol-2-yl propyl.

The term “heterocyclylalkoxy” refers to alkyl groups as defined herein in which a hydrogen or carbon bond of an alkyl group as defined herein is replaced with a bond to a heterocyclyl group as defined herein and the alkyl group is attached to an oxygen.

Representative heterocyclylalkoxy groups include, but are not limited to, —O—(CH₂)_{q}heterocyclyl, wherein q is an integer from 1 to 5. In some embodiments, heterocyclylalkoxy groups include —O—(CH₂)_{q}morpholinyl such as —O—CH₂CH₂-morpholine.

The term “heteroarylalkyl” refers to alkyl groups as defined herein in which a hydrogen or carbon bond of an alkyl group is replaced with a bond to a heteroaryl group as defined herein.

The term “alkoxy” refers to an oxygen atom connected to an alkyl group, including a cycloalkyl group, as are defined herein. Examples of linear alkoxy groups include, but are not limited to, methoxy, ethoxy, propoxy, butoxy, pentyloxy, hexyloxy, and the like. Examples of branched alkoxy include, but are not limited to, isopropoxy, sec-butoxy, tert-butoxy, isopentyloxy, isohexyloxy, and the like. Examples of cyclic alkoxy include, but are not limited to, cyclopropyloxy, cyclobutyloxy, cyclopentyloxy, cyclohexyloxy, and the like. An alkoxy group can include one to about 12-20 or about 12-40 carbon atoms bonded to the oxygen atom, can further include double or triple bonds, and can also include heteroatoms. For example, an allyloxy group is an alkoxy group within the meaning herein. A methoxyethoxy group is also an alkoxy group within the meaning herein, as is a methylenedioxy group in a context where two adjacent atoms of a structure are substituted therewith.

The terms “amine,” “amine group,” “amino,” and “amino group” refer to a substituent of the form —NH₂, —NHR, —NR₂, or —NR₃⁺, wherein each R is defined herein, and protonated forms of each, except for —NR₃⁺, which cannot be protonated. Accordingly, any compound substituted with an amino group can be viewed as an amine. An “amino group” within the meaning herein can be a primary, secondary, tertiary, or quaternary amino group.

An “alkylamino” group includes a monoalkylamino, dialkylamino, and trialkylamino group. An example of a “alkylamino” is —NH-alkyl and —N(alkyl)₂.

An example of a “cycloalkylamino” group is —NH-cycloalkyl and —N(cycloalkyl)₂.

An example of a “cycloalkyl heterocycloamino” group is —NH-(heterocyclo cycloalkyl), wherein the heterocyclo group is attached to the nitrogen and the cycloalkyl group is attached to the heterocyclo group.

An example of a “heterocyclo cycloamino” group is —NH-(cycloalkyl heterocycle), wherein the cycloalkyl group is attached to the nitrogen and the heterocyclo group is attached to the cycloalkyl group.

The term “amido” refers to a group of the formula —C(O)NR₂, wherein R is defined herein.

The terms “halo,” “halogen,” and “halide” group, by themselves or as part of another substituent, mean, unless otherwise stated, a fluorine, chlorine, bromine, or iodine atom.

The term “haloalkyl” group includes mono-halo alkyl groups, poly-halo alkyl groups, wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, 1,2-dichloroethyl, 1,3-dibromo-3,3-difluoropropyl, perfluorobutyl, —CF(CH₃)₂ and the like.

Values expressed in a range format should be interpreted in a flexible manner to include not only the numerical values explicitly recited as the limits of the range, but also to include all the individual numerical values or sub-ranges encompassed within that range as if each numerical value and sub-range were explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. In addition, it is to be understood that the phraseology or terminology employed herein, and not otherwise defined, is for the purpose of description only and not of limitation. Any use of section headings is intended to aid reading of the document and is not to be interpreted as limiting. Further, information that is relevant to a section heading can occur within or outside of that particular section. Furthermore, all publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

In the methods described herein, the steps can be carried out in any order without departing from the principles of the invention, except when a temporal or operational sequence is explicitly recited.

Furthermore, specified steps can be carried out concurrently unless explicit claim language recites that they be carried out separately. For example, a claimed step of doing X and a claimed step of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more.

The term “substantially no” as used herein refers to less than about 30%, 25%, 20%, 15%, 10%, 5%, 3%, 2%, 1%, 0.5%, 0.1%, 0.05%, 0.001%, or at less than about 0.0005% or less or about 0% or 0%.

Those skilled in the art will appreciate that many modifications to the embodiments described herein are possible without departing from the spirit and scope of the present disclosure. Thus, the description is not intended and should not be construed to be limited to the examples given but should be granted the full breadth of protection afforded by the appended claims and equivalents thereto. In addition, it is possible to use some of the features of the present disclosure without the corresponding use of other features. Accordingly, the foregoing description of or illustrative embodiments is provided for the purpose of illustrating the principles of the present disclosure and not in limitation thereof and can include modification thereto and permutations thereof.

What is claimed is:

1. A therapeutic method of treating an impulse-control disorder comprising administering to an individual afflicted with an impulse-control disorder an amount of at least one glucagon-like-peptide-1 (GLP-1) effective to reduce or eliminate at least one of the symptoms of the impulse-control disorder.

2. The therapeutic method of claim 1, wherein the impulse-control disorder is compulsive sexual disorder.

3. The therapeutic method of claim 1 wherein the impulse-control disorder is pathological gambling.

4. The therapeutic method of claim 1 wherein the impulse-control disorder is intermittent explosive disorder.

5. The therapeutic method of claim 1 wherein the impulse-control disorder is compulsive shopping.

6. The therapeutic method of claim 1 wherein the impulse-control disorder is pyromania.

7. The therapeutic method of claim 1 wherein the impulse-control disorder is kleptomania.

8. The therapeutic method of claim 1 wherein the agonist is administered orally.

9. The therapeutic method of claim 1 wherein the agonist is administered parenterally.

10. The therapeutic method of claim 9, wherein the agonist is administered by injection or infusion.

11. The therapeutic method of claim 9, wherein the agonist is administered by means of a controlled release dosage form.

12. The therapeutic method of claim 11, wherein the agonist is administered by means of a transdermal patch.

13. The therapeutic method of claim 1, wherein the agonist is administered by inhalation.

14. The method of claim 1 wherein the agonist is tirzepatide, dulaglutide, exenatide (weekly and twice-daily), exenatide, semaglutide, liraglutide or lixisenatide or salts thereof or combinations thereof.
